(19) United States
(12) Patent Application Publication
(10) Pub. No.: US 2024/0182834 A1
(43) Pub. Date: Jun. 6, 2024

(54) RECONFIGURABLE BIOPROCESSING SYSTEM

(71) Applicant: CYTIVA SWEDEN AB, Uppsala (SE)

(72) Inventors: Andreas Lundin, Uppsala (SE); Mats Rimmo, Uppsala (SE)

(21) Appl. No.: 18/553,821

(22) PCT Filed: Apr. 25, 2022
(86) PCT No.: PCT/EP2022/060892

§ 371 Date: Oct. 3, 2023

(30) Foreign Application Priority Data
Apr. 26, 2021 (GB) 2105909.2

(51) Int. Cl. C12M 1/00; C12M 1/34; C12M 1/36; C12M 3/00

(52) U.S. Cl. CPC C12M 29/04; C12M 23/40; C12M 23/42; C12M 23/44; C12M 41/30; C12M 41/48

(57) ABSTRACT

The present disclosure relates to a reconfigurable bioprocessing system (80) operable to perform a predetermined set of bioprocessing operations within the bioprocessing system (80), the bioprocessing system (80) comprising: a base unit (60) comprising a plurality of valve actuators, wherein at least one of the plurality of valve actuators is configured to releasably engage with a membrane valve cassette (200, 300, 400) that is removably attachable to the base unit (60), and wherein the at least one of the plurality of valve actuators is actuatable to regulate fluid flow through the membrane valve cassette (200, 300, 400); and a control system (20) operable to selectively actuate respective ones of the plurality of valve actuators to provide a valve configuration within the membrane valve cassette (200, 300, 400) to enable the base unit (60) and the membrane valve cassette (200, 300, 400) together to perform one of the predetermined set of bioprocessing operations.

### FIELD

The present disclosure relates to a reconfigurable bioprocessing system operable to perform a predetermined set of bioprocessing operations. The present disclosure also relates to one or more removably attachable cassettes that may be used to perform the set of bioprocessing operations.

### BACKGROUND

Bioprocessing systems such as liquid chromatography systems typically require effective liquid handling in order to carry out processes under controlled and contained conditions. This may involve aseptic handling in closed fluid handling systems and the use of pre-sterilised components.

Recently, modular bioprocessing systems based on single-use flow paths have been developed. Single-use flow path components eliminate the time- and labour-consuming pre- and post-cleaning of the wetted flow path. This increases the overall process efficiency and reduces cost. The elimination of equipment cleaning and any associated cleaning validation processes also greatly reduces the risk of cross-contamination between different batches being processed using the bioprocessing system.

The single-use components are implemented as modular consumables that are intended to be disposed of after a process run. Examples of modular bioprocessing systems include the systems described in WO 2015/095658, WO 2018/158273, WO 2019/057936, WO 2019/057937 and WO 2020/065040.

WO 2015/095658 describes a biological liquid handling system that includes a valve arrangement that can be remotely actuated by a pneumatic actuation system. The valve arrangement controls the flow of fluid in a single-use flow path. The valve arrangement includes membrane valves that are pneumatically actuated by application of pneumatic pressure from a pneumatic control system, via pneumatic conduits. Different valve arrangements are used for different liquid processing tasks (for example, different numbers, physical arrangements, sizes and functionalities of the valves).

WO 2018/158273 describes a bioprocessing cassette in which a fluid path through the cassette can be controlled using valves that route fluid either through a component of the cassette or to an outlet. The valves include exposed valve stems that allow the valves to be manually actuated in order to control the fluid path through the cassette.

WO 2020/065040 describes a biological fluid processing system that aims to provide increased configurability over prior systems. To provide increased configurability, the fluid processing system implements a fluid processing interface in between a processing control element and a fluid processing device. The fluid processing devices that can be used in the system can differ from one another (e.g. with different configurations or capacities), and the fluid processing interface provides the physical and mechanical interfaces required in order to allow corresponding fluid processing device to interface with the processing control element.

WO 2019/057936 describes a chromatography apparatus that aims to provide increased configurability over prior systems. To provide increased configurability, the chromatography apparatus implements a plurality of modular flow path components that can be mounted to a housing of the chromatography apparatus. The modular components can be connected together to form a flow path. The flow path components are provided in distinct modules so that the modules can be repositioned and interchanged to suit a particular procedure. Fluid flow through the flow path is controlled by a valve unit (as described in WO 2019/057937), which includes a plurality of membrane valves, each actuated by a piston within the valve unit.

While modular bioprocessing systems are known, the industry is always seeking further improvements to such modular bioprocessing systems, particularly to provide modular bioprocessing systems with increased reconfigurability.

### SUMMARY

This summary introduces concepts that are described in more detail in the detailed description. It should not be used to identify essential features of the claimed subject matter, nor to limit the scope of the claimed subject matter.

According to a first aspect, there is provided a reconfigurable bioprocessing system operable to perform a predetermined set of bioprocessing operations within the bioprocessing system, the bioprocessing system comprising: a base unit comprising a plurality of valve actuators, wherein at least one of the plurality of valve actuators is configured to releasably engage with a membrane valve cassette that is removably attachable to the base unit, and wherein the at least one of the plurality of valve actuators is actuatable to regulate fluid flow through the membrane valve cassette; and a control system operable to selectively actuate respective ones of the plurality of valve actuators to provide a valve configuration within the membrane valve cassette to enable the base unit and the membrane valve cassette together to perform one of the predetermined set of bioprocessing operations.

The reconfigurable bioprocessing system of the first aspect allows a fluid flow path to be easily reconfigured to provide a valve configuration suitable for a particular bioprocessing operation. In particular, the fluid flow path can be easily reconfigured by actuation of the actuators using the control system, to provide a different valve configuration. In addition, by providing a base unit with at least one actuator that releasably engages with a membrane valve cassette that is removably attachable to the base unit, a common interface for actuating valves in a removably attachable standard membrane valve cassette is provided. This common interface provides increased reconfigurability of the flow path because different types of membrane valve cassettes can be utilised with the base unit, depending on the bioprocessing operation being carried out. The flow path can be easily reconfigured by the control system, which is capable of providing valve configurations that enable a set of bioprocessing operations to be carried out. Consequently, the bioprocessing system provides reconfigurability of the flow path to allow the system to carry out a particular bioprocessing operation from a set of bioprocessing operations, and reconfigurability of the flow path used for the particular bioprocessing operation by providing a different valve configuration.

By providing such a reconfigurable bioprocessing system not only can a single base unit be used to provide multiple different types of bioprocessing operation, but also such a bioprocessing system can be operated by less skilled users/operators than are conventionally required, as will be apparent from the description given below.

According to a second aspect, there is provided a membrane valve cassette for a reconfigurable bioprocessing system according to the first aspect, wherein the membrane valve cassette is configured to removably attach to a base unit of the reconfigurable bioprocessing system.

As noted above, the base unit of the bioprocessing system provides a common interface allowing different types of membrane valve cassette to be utilised with the base unit, in order to carry out each of a set of bioprocessing operations. The membrane valve cassette of the second aspect may therefore include flow path components that are specific to the particular bioprocessing operation being carried out.

According to a third aspect, there is provided a flow path pressure monitoring cassette for a bioprocessing system, wherein the flow path pressure monitoring cassette is removably attachable to the bioprocessing system, the flow path pressure monitoring cassette comprising: one or more conduits; and an attachment mechanism configured to engage a pressure sensing device for sensing a pressure of fluid flowing through at least one of the one or more conduits, wherein the attachment mechanism is configured to seal around a sensing area of the pressure sensing device when the pressure sensing device is engaged with the attachment mechanism.

According to a fourth aspect, there is provided a flow path air trap cassette for a bioprocessing system, wherein the flow path air trap cassette is removably attachable to the bioprocessing system, the flow path air trap cassette comprising: one or more conduits; an air trap in fluidic communication with at least one of the one or more conduits, wherein the air trap is configured to capture air present in fluid flowing through the at least one of the one or more conduits; and a sensor configured to detect a level of fluid within the air trap.

According to a fifth aspect, there is provided a configurable flow path cassette for a bioprocessing system, wherein the configurable flow path cassette is removably attachable to the bioprocessing system, the configurable flow path cassette comprising: a plurality of ports comprising a first port, a second port, a first group of ports, and a second group of ports, wherein the first group of ports is in fluidic communication with the first port; and a plurality of valve interfaces that are actuatable to provide a first configuration in which the second group of ports is in fluidic communication with the first port and a second configuration in which the second group of ports is in fluidic communication with the second port.

According to a sixth aspect, there is provided a conductivity sensing device for a bioprocessing system, the conductivity sensing device comprising: a conductivity sensor; a sealing portion having an opening configured to receive the conductivity sensor; and a casing comprising an attachment mechanism configured to attach the conductivity sensing device to the bioprocessing system; wherein the sealing portion is configured to form a seal around the conductivity sensor when the conductivity sensing device is attached to the bioprocessing system.

The cassettes according to the third to fifth aspects and the device according to the sixth aspect allow the holdup volume within a flow path of the bioprocessing system to be minimised. In particular, the third aspect provides for the detection of a pressure of a fluid flow in a removably attachable cassette that is used to perform a bioprocessing operation. In addition, the fourth aspect provides for integration of an air trap into a removably attachable cassette that is used to perform a bioprocessing operation. Further, the fifth aspect provides for configurability of a number of fluid inlets or outlets provided by a removably attachable cassette that is used to perform a bioprocessing operation. Lastly, the sixth aspect provides for the detection of a conductivity of a fluid flow in a removably attachable cassette that is used to perform a bioprocessing operation.

The provision of the above functionality in removably attachable cassettes used in bioprocessing operations allows various flow path operations (such as a pressure or conductivity measurement, air capture, or routing of the fluid flow) to be performed within the removably attachable cassette, and without having to route the fluid flow through a separate flow path component that provides the flow path operation. Performing the various flow path operations within the removably attachable cassette minimises the length of tubing used to connect flow path components, meaning that the holdup volume of fluid in the flow path is, in turn, minimised.

According to a seventh aspect, there is provided a flow path kit for a bioprocessing system, the flow path kit comprising at least one of: a membrane valve cassette according to the second aspect; a flow path pressure monitoring cassette according to the third aspect; a flow path air trap cassette according to the fourth aspect; and a configurable flow path cassette according to the fifth aspect; wherein the flow path kit optionally further comprises a conductivity sensing device according to the sixth aspect.

### BRIEF DESCRIPTION OF FIGURES

Specific embodiments are described below by way of example only and with reference to the accompanying drawings, in which:

FIG. 1A is a schematic diagram of a base unit of a reconfigurable bioprocessing system.

FIG. 1B is a schematic diagram of a reconfigurable bioprocessing system including the base unit shown in FIG. 1A and a flow path kit.

FIG. 1C is a schematic diagram of the flow path kit used in the reconfigurable bioprocessing system shown in FIG. 1B.

FIG. 2A is a schematic diagram of a control system of the reconfigurable bioprocessing system shown in FIG. 1B.

FIG. 2B is a flow diagram showing operation of the control system shown in FIG. 2A.

FIG. 3 is a schematic diagram of a plurality of valve actuators of the base unit shown in FIG. 1A.

FIG. 4 is a cross-section through one of the actuators shown in FIG. 3.

FIGS. 5A and 5B are schematic diagrams of a membrane valve cassette comprising an array of valve interfaces.

FIG. 6 is a cross-section through one of the valve interfaces shown in FIGS. 5A and 5B.

FIG. 7 is a diagram showing the membrane valve cassette shown in FIGS. 5A and 5B attached to the plurality of actuators shown in FIG. 3.

FIG. 8 is an isometric view of the membrane valve cassette shown in FIGS. 5A and 5B.

FIG. 9 is a cross-section through the membrane valve cassette shown in FIGS. 5A and 5B.

FIG. 10A is a schematic diagram of a membrane valve cassette configured as a configurable flow path cassette.

FIG. 10B is an isometric view of the configurable flow path cassette schematically illustrated in FIG. 10A.

FIG. 10C is a schematic diagram of an additional membrane valve cassette configured as a configurable flow path cassette.

FIG. 11 is a front view of a membrane valve cassette configured as a system valve cassette.

FIG. 12 is a schematic diagram of the system valve cassette shown in FIG. 11.

FIG. 13 is a schematic diagram of an air trap of the system valve cassette shown in FIG. 11.

FIG. 14 is a schematic diagram of a pressure sensing device attached to the system valve cassette shown in FIG. 11.

FIG. 15 is an exploded view of a conductivity sensing device configured to attach to system valve cassette shown in FIG. 11.

FIG. 16 is a schematic diagram showing the conductivity sensing device in FIG. 15 in assembled form.

FIG. 17 is a schematic diagram showing the conductivity sensing device in FIG. 15 attached to the system valve cassette shown in FIG. 11.

FIG. 18 is a flowchart of a method of manufacturing a conductivity sensing device.

FIG. 19 is a flowchart of a method for reconfiguring a bioprocessing system.

FIG. 20 is a partial cross section of an embodiment of a system according to the present invention that includes a valve actuation unit and a membrane valve cassette coupled thereto.

### DETAILED DESCRIPTION

Implementations of the present disclosure are explained below with particular reference to bioprocessing systems. It will be appreciated, however, that the implementations described below are applicable to other fluid handling systems.

It will be understood that in the following detailed description, where two components are described as being in “fluidic communication”, fluid is permitted to flow between the two components, wherein the flow of fluid may be controlled by a valve.

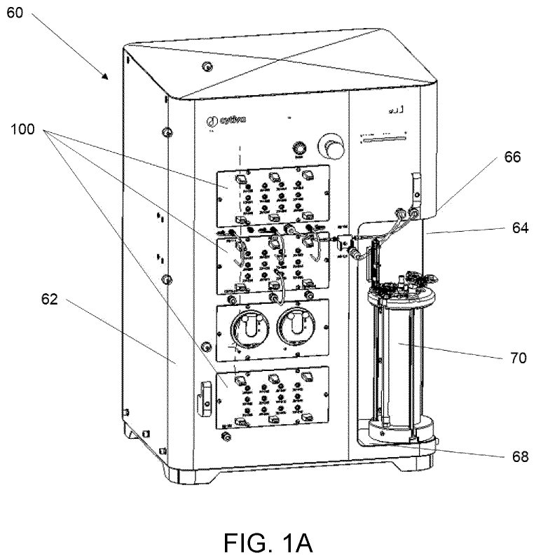

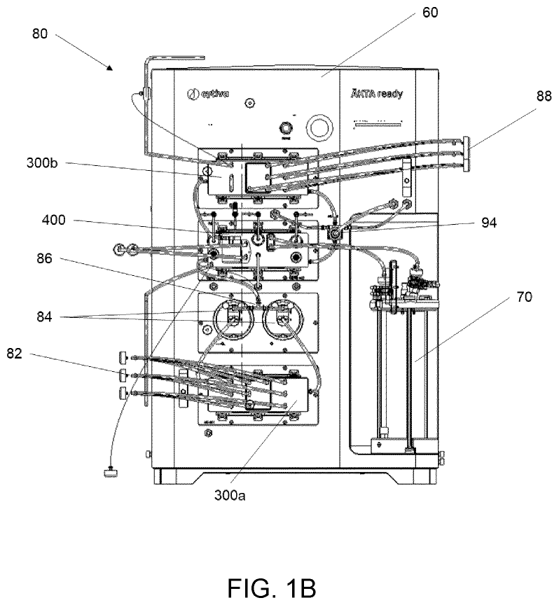

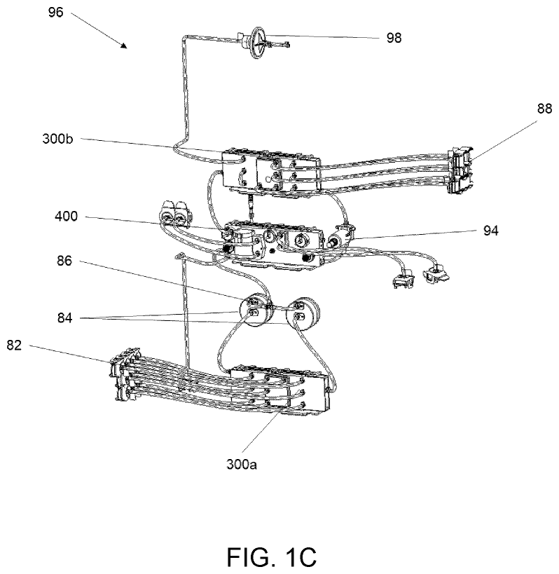

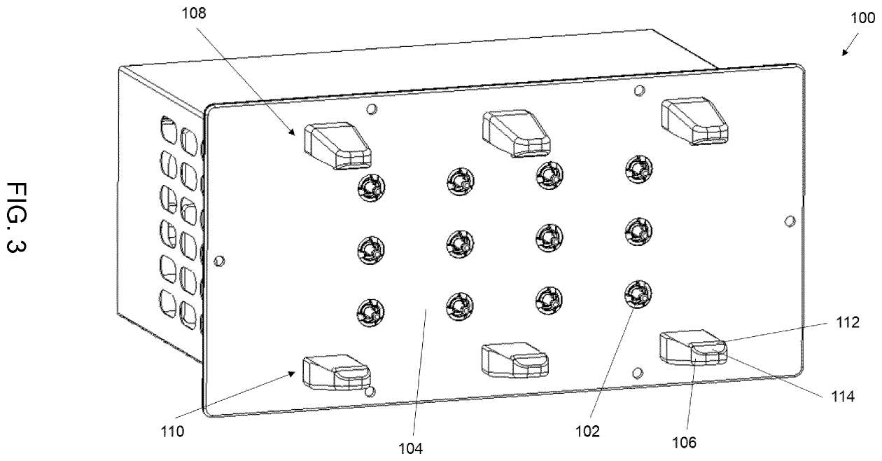

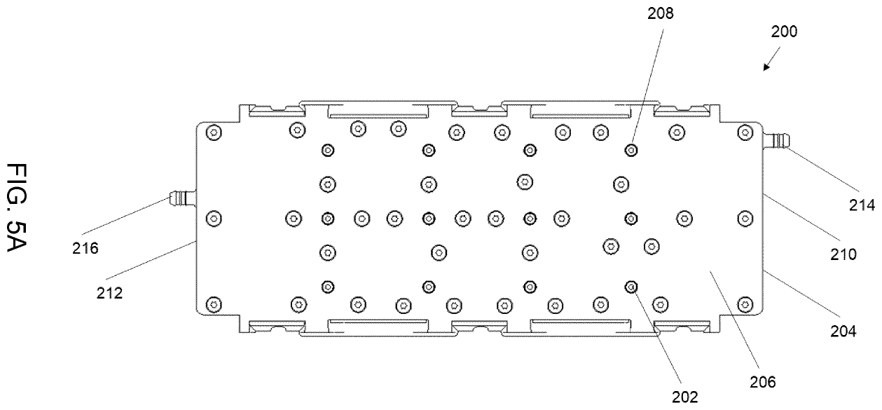

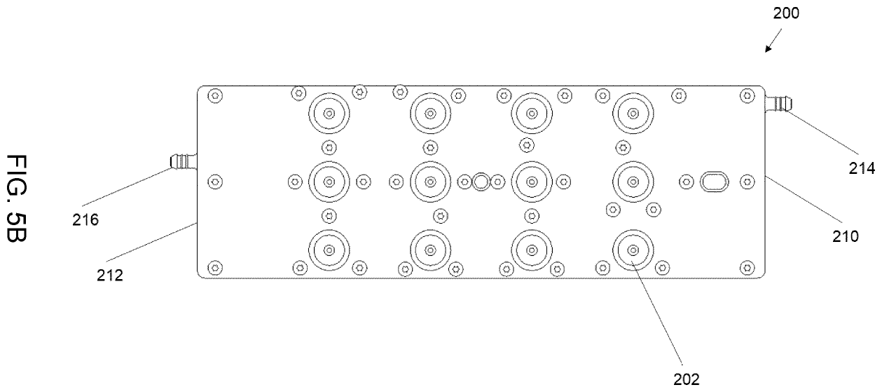

The present disclosure relates to a reconfigurable bioprocessing system (illustrated schematically in FIG. 1B). The reconfigurable bioprocessing system is operable to perform a predetermined set of bioprocessing operations (e.g. a plurality of bioprocessing operations) within the bioprocessing system. The bioprocessing system comprises a base unit (illustrated schematically in FIG. 1A). The base unit comprises a plurality of valve actuators (as shown in FIG. 3). At least one of the plurality of valve actuators is configured to releasably engage with a membrane valve cassette (illustrated schematically in FIGS. 5A and 5B). The membrane valve cassette is removably attachable to the base unit. The at least one of the plurality of valve actuators is actuatable to regulate fluid flow through the membrane valve cassette. The bioprocessing system also comprises a control system operable to selectively actuate respective ones of the plurality of valve actuators to provide a valve configuration within the membrane valve cassette to enable the base unit and the membrane valve cassette together to perform one of the predetermined set of bioprocessing operations.

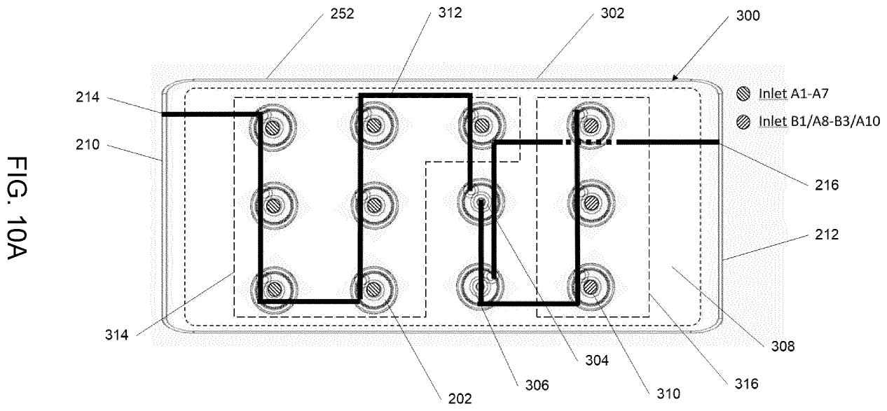

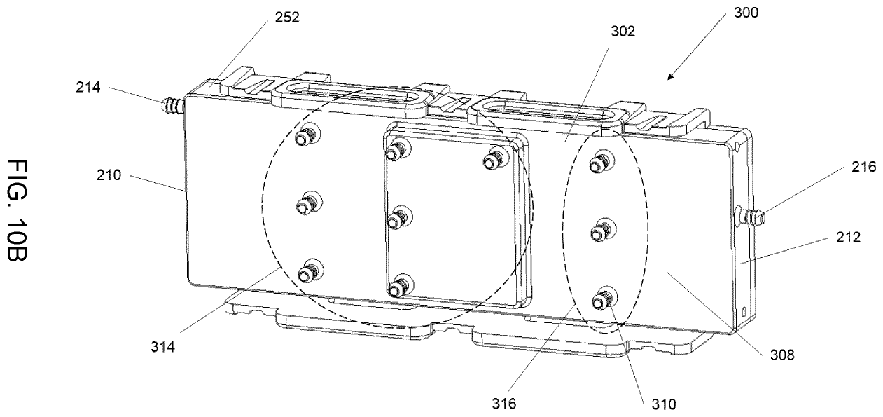

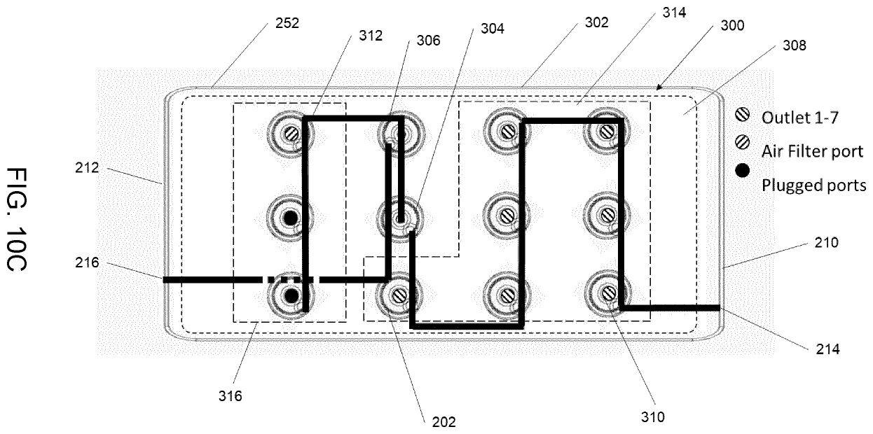

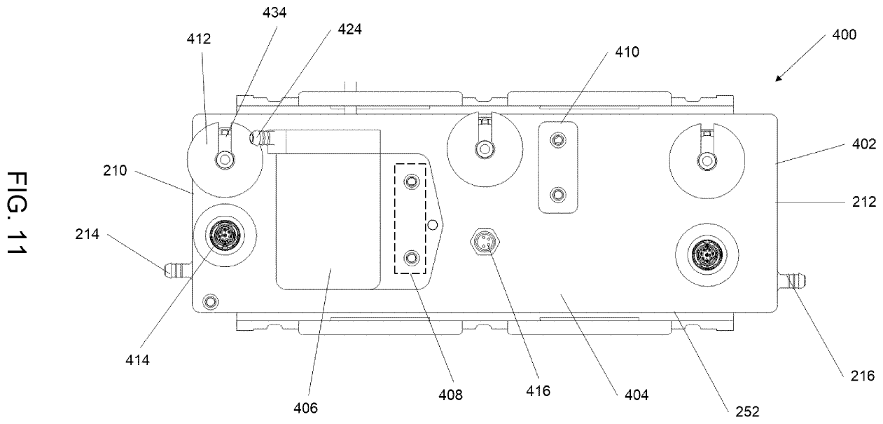

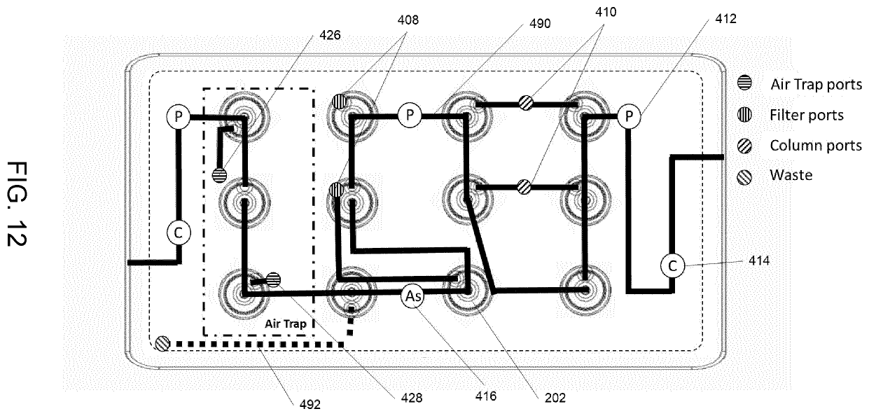

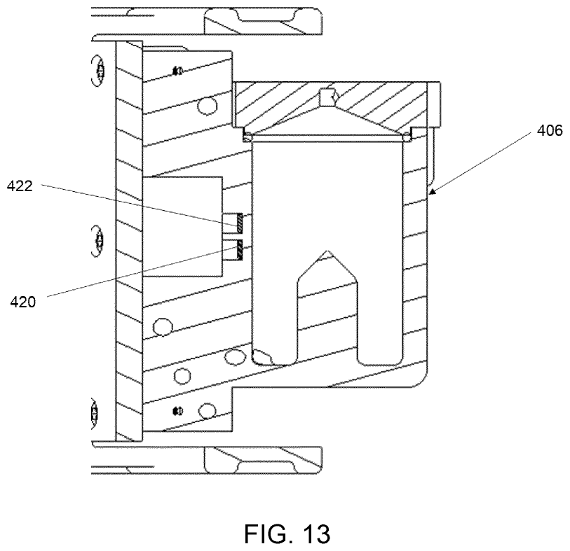

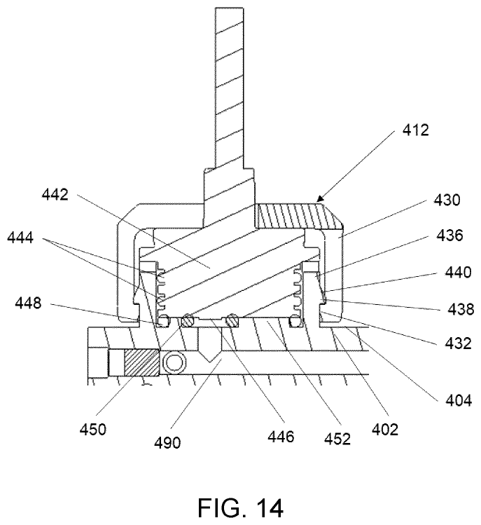

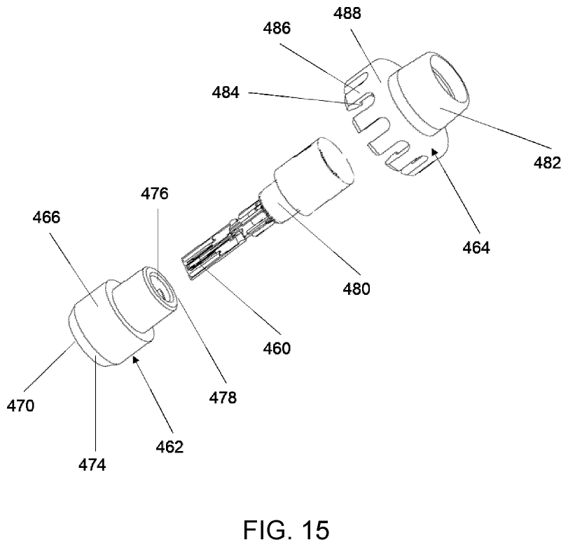

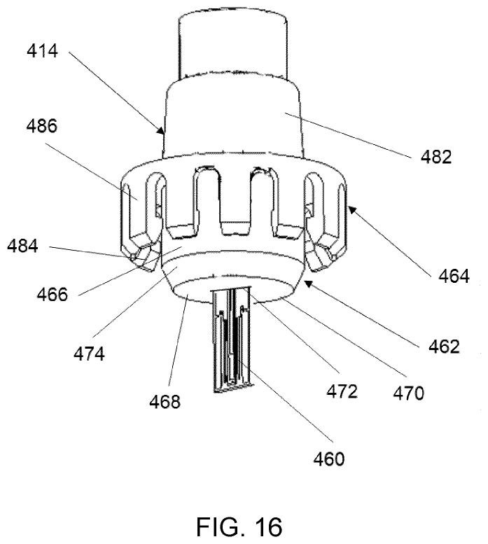

A number of different types of membrane valve cassette may be removably attached to the base unit for performing the predetermined set of bioprocessing operations. A first type of membrane valve cassette that may be removably attached to the base unit is a configurable flow path cassette (illustrated schematically in FIGS. 10A to 10C). A second type of membrane valve cassette that may be removably attached to the base unit is a flow path air trap cassette comprising an air trap (e.g. the air trap illustrated schematically in FIG. 13). A third type of membrane valve cassette that may be removably attached to the base unit is a flow path pressure monitoring cassette, which may be used in combination with a pressure sensing device (e.g. as illustrated schematically in FIG. 14). These types of membrane valve cassette may also be configured for attachment to a conductivity sensing device (e.g. as illustrated schematically in FIGS. 15 and 16). Flow path components of the membrane valve cassette types described above may be implemented in combination in a membrane valve cassette. For example, a fourth type of membrane valve cassette that may be removably attached to the base unit is a system valve cassette (illustrated schematically in FIGS. 11 and 12) that comprises an air trap and is configured for attachment to a plurality of pressure sensing devices and conductivity sensing devices. It will be appreciated that the example membrane valve cassettes described herein are not limiting, and additional types of membrane valve cassette that may be removably attached to the base unit may include other combinations of the flow path components described herein.

FIG. 1A is a schematic diagram of a base unit 60 of a reconfigurable bioprocessing system operable to perform a predetermined set of bioprocessing operations. For example, the bioprocessing system may be operable to perform one or more of: a chromatography operation, a mixing operation, and/or a filtration operation (e.g. tangential flow filtration (TFF)). The predetermined set may additionally, or alternatively, include operation using the same type of bioprocessing operation but accommodating different operating flow rates, processing volumes and/or system hold-up volumes that may be required. The predetermined set may comprise operation of more or less complex flow paths within one type of unit operation.

However, it is to be understood that such a set of bioprocessing operations may, additionally or alternatively, include one or more different types of bioprocessing operation where the operating parameters thereof are changed. For example, a chromatography operation may be chosen and one or more different types of such a chromatography operation and/or sets of operating parameters may be selected (e.g. for high-performance liquid chromatography (HPLC), reversed-phase chromatography (RPC), size-exclusion chromatography, ion-exchange chromatography, and/or simulated moving bed (SMB) chromatography, etc.)

In other examples, unit operations other than chromatography may be chosen, such as filtration, fluid conditioning (e.g. including mixing and/or reaction steps etc.) With filtration operations, different types of filtration may be selected, such as normal flow filtration, virus filtration, crossflow filtration with retentate recirculation (multi-pass filtration) and/or crossflow filtration in single-pass filtration configurations.

As shown in FIG. 1A, the base unit 60 comprises a housing 62 in which three valve actuation units 100 are mounted. Each valve actuation unit 100 includes a plurality of valve actuators. Each valve actuation unit 100 is configured to releasably engage (via the plurality of valve actuators) with a corresponding membrane valve cassette. In particular, the plurality of valve actuators of each valve actuation unit 100 are actuatable to regulate fluid flow through the corresponding membrane valve cassette.

As shown in FIG. 1A, the housing 62 includes an elongate recess 64. The recess 64 is provided at a corner 66 of the housing 62. In particular, the housing 62 is substantially cuboidal and the recess 64 provided in the corner 66 of the housing 62 is also substantially cuboidal. The recess 64 is configured to accommodate a chromatography column 70, and comprises a base 68 on which the chromatography column 70 may be supported. By positioning the chromatography column 70 in this way, the chromatography column 70 is provided within the footprint of the housing 62.

Positioning the chromatography column 70 on the base 68 within the recess 64 also means that the column 70 does not need to be mounted separately on a surface (e.g. a workbench) on which the housing 62 is positioned. This means that the chromatography column 70 can be positioned closer to the membrane valve cassette(s), thereby allowing a shorter length of tubing to be used to connect the membrane valve cassette(s) to the chromatography column 70. Reducing the length of tubing means that less fluid is held within the tubing, which reduces the hold-up volume of the fluid being processed (i.e. the total volume of fluid within a flow path of the bioprocessing system).

To allow for attachment of smaller sized chromatography columns, the recess 64 may also include fixings provided on a side wall of the recess 64, which allow a smaller sized chromatography column to be held above the base within the recess 64. Fixing a smaller sized chromatography column to a side wall of the recess 64 (instead of supporting it using the base 68) allows the top of the chromatography column to be positioned closer to the membrane valve cassette(s), thereby allowing a shorter length of tubing to be used for connecting the membrane valve cassette(s) to the column.

FIG. 1B shows an assembled bioprocessing system 80 comprising the base unit 60 (including three valve actuation units 100), along with a chromatography column 70 and a flow path kit 96 comprising three membrane valve cassettes, a pair of pumps 84, a pH/UV sensor device 94, and tubing connecting the various components. The flow path kit 96 is shown separately in FIG. 1C. Together, the chromatography column 70 and flow path kit 96 form a flow path.

The bioprocessing system 80 also includes a control system (not shown in FIG. 1B). The control system is operable to selectively actuate respective ones of the plurality of valve actuators in each valve actuation unit 100. The activation of the plurality of valve actuators provides a valve configuration within the membrane valve cassette. Different valve configurations allow the base unit 60 (and attached membrane valve cassette(s)) to perform a bioprocessing operation. The valve configuration therefore enables the base unit 60 and the membrane valve cassette together to perform one of the bioprocessing operations in the set of bioprocessing operations that the bioprocessing system is operable to perform.

In use, a first membrane valve cassette in the form of a first configurable flow path cassette (indicated in FIG. 1B as 300a and described in more detail with reference to FIGS. 10A-10C) provides fluidic communication between inlets 82 and the pair of pumps 84. The pumps 84 drive the flow of fluid within the flow path. As described below in relation to FIGS. 10A and 10B, the first configurable flow path cassette 300a may be configured so that different numbers of inlets may be provided to each of the pumps 84. The configuration of the flow through the first configurable flow path cassette 300a is determined by the valve configuration provided by the selective actuation of the plurality of valve actuators in the valve actuation unit 100 to which the first configurable flow path cassette 300a is attached.

The pumps 84 are fluidically connected to a T-section 86, which allows for mixing of different fluids (where, for example, a different fluid is inlet to each pump 84), or for increased fluid flow (where, for example, the same fluid is inlet to each pump 84). Mixing the fluids allows a split (or gradient) of different fluids to be provided, where each fluid is pumped by a different pump 84. For example, this allows the relative proportions of different fluids to be adjusted during a bioprocessing operation.

The outlet from the T-section 86 is fluidically connected to a second membrane valve cassette in the form of a system valve cassette 400 (described in more detail with reference to FIGS. 11-14). As described below, the system valve cassette 400 allows fluid to be optionally routed through (i) an air trap; (ii) a filter connected to a first pair of ports in the system valve cassette 400; and (iii) the chromatography column 70, which is connected to a second pair of ports in the system valve cassette 400. The routing of fluid through these components of the system valve cassette 400 is determined by the valve configuration provided by the selective actuation of the plurality of valve actuators in the valve actuation unit 100 to which the system valve cassette 400 is attached.

In particular, the valve configuration may allow the pressure of fluid flowing in conduits of the system valve cassette 400 to be measured using pressure sensing devices attached to the system valve cassette 400. In addition, the valve configuration may allow the salt level of fluid flowing in conduits of the system valve cassette 400 to be measured using conductivity sensing devices attached to the system valve cassette 400. Further, the valve configuration may allow the level of air in fluid flowing in a conduit of the system valve cassette 400 to be measured using an air detection sensor of the system valve cassette 400.

In the example shown in FIG. 1B, an air outlet of the air trap of the system valve cassette 400 is connected to a third membrane valve cassette in the form of a second configurable flow path cassette (indicated in FIG. 1B as 300b and described in more detail with reference to FIGS. 10A-10C). A valve interface in the second configurable flow path cassette 300b is used to open a vent outlet to route the air outlet from the air trap to an air filter (shown in FIG. 1C).

The fluid outlet from the system valve cassette 400 (e.g. the fluid return from the chromatography column 70) is also routed through the pH/UV sensor device 94 and subsequently to the second configurable flow path cassette 300b. The pH/UV sensor device 94 (as best shown in FIG. 1C) includes a pH sensor to measure the pH of the fluid outlet from the system valve cassette 400. A chromatography process may involve releasing proteins from the chromatography column 70 by controlling the pH of fluid flowing through the column. The pH sensor therefore allows for the pH of the fluid flowing through the column to be monitored. The pH/UV sensor device 94 also includes connections for attachment of optical fibres to the device, which allows the fluid passing through the pH/UV sensor device 94 to be illuminated using ultraviolet, visible or infrared radiation. The pH/UV sensor device 94 includes a UV sensor which measures the amount of radiation passing through the fluid flow. This, in turn, allows the amount of radiation absorbed by the fluid flow to be determined, which allows the presence of sample molecules in the fluid flow that absorb radiation at the wavelength used for the measurement to be determined.

As described below in relation to FIG. 10C, the second configurable flow path cassette 300b may be configured so that different numbers of outlets are be provided.

Consequently, the fluid outlet from the system valve cassette 400 may be routed to a number of different outlets 88 configured by the second configurable flow path cassette 300b. The configuration of the flow through the second configurable flow path cassette 300b is determined by the valve configuration provided by the selective actuation of the plurality of valve actuators in the valve actuation unit 100 to which the second configurable flow path cassette 300b is attached.

FIG. 1C is a schematic diagram of the flow path kit 96 shown in FIG. 1B. The flow path kit 96 is configured for attachment to the base unit 60 shown in FIG. 1A. The flow path kit 96 includes the configurable flow path cassettes 300a and 300b, the system valve cassette 400, the pair of pumps 84, the pH/UV sensor device 94, and tubing connecting the various components. The flow path kit 96 also includes an air filter 98 configured to filter the air outlet from the air trap of the system valve cassette 400.

Various examples described herein may use single-use technology. For example, the flow path kit 96 may be a single-use flow path kit. Such examples may thus include one or more component(s), such as aseptic connectors, etc., that can be pre-sterilized. For example, gamma sterilisation may be used and various materials that are compatible therewith can be provided.

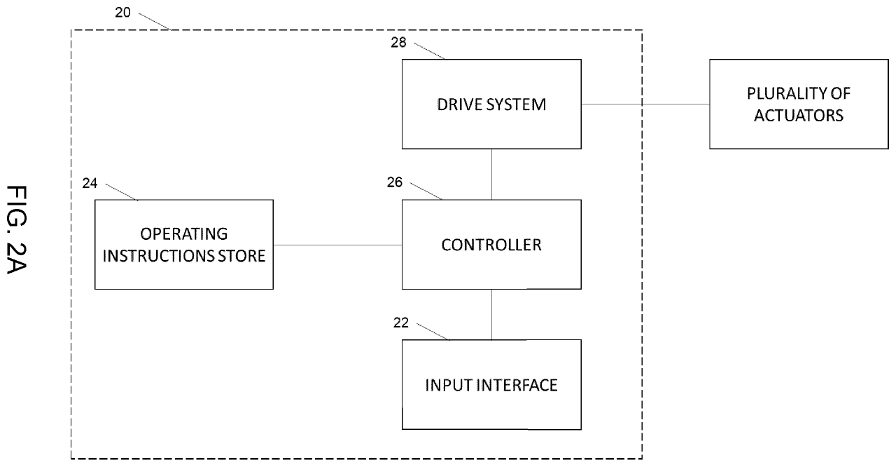

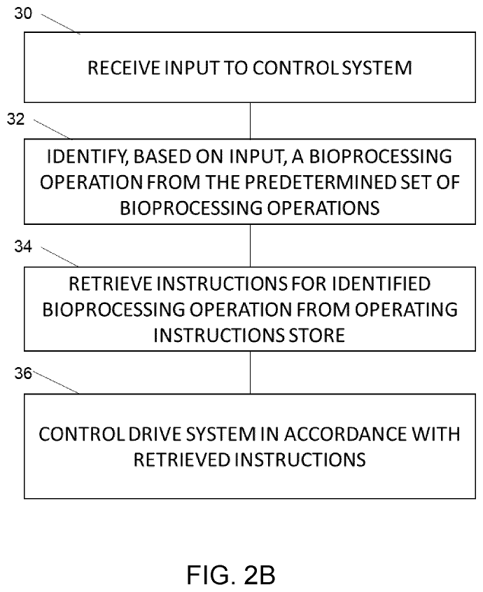

FIG. 2A is a schematic diagram of a control system 20 that can be used with the reconfigurable bioprocessing system 80 in FIG. 1B. As shown in FIG. 2A, the control system 20 includes an input interface 22. The input interface 22 is configured to receive identifiers of the components that are attached to the base unit 60. For example, the input interface 22 is configured to receive identifiers of the flow path kit 96 and/or the individual membrane valve cassettes attached to the base unit 60. The input interface 22 may receive the identifiers via an RFID chip (or other automated identifier providing feature) on each flow path kit and/or cassette that provides the identifier to the input interface 22. Alternatively or additionally, the identifiers may be input to the input interface 22 manually by an operator of the bioprocessing system 80 (e.g. via a user interface of the bioprocessing system 80).

The control system 20 also includes an operating instructions store 24 configured to store operating instructions for a predetermined set of bioprocessing operations. For example, the operating instructions store 24 may store operating instructions for one or more chromatography operations, one or more mixing operations (allowing fluid gradients to be provided), and/or one or more filtration operations.

The control system 20 further includes a controller 26 configured to identify a bioprocessing operation from the set of bioprocessing operations, and to retrieve the operating instructions for the identified bioprocessing operation from the operating instructions store 24. The controller 26 may identify the bioprocessing operation based on the identifiers of the flow path kit and/or membrane valve cassettes received by the input interface 22. The controller 26 may additionally or alternatively identify the bioprocessing operation based on a manual input of a desired bioprocessing operation to the input interface 22 (e.g. via a user interface of the bioprocessing system 80). Once the controller 26 has identified the bioprocessing operation, the controller 26 retrieves the operating instructions for the identified bioprocessing operation from the operating instructions store 24.

The control system 20 further includes a drive system 28 that receives control signals from the controller 26. In particular, the controller 26 reads the retrieved operating instructions and provides control signals to the drive system 28 in accordance with the operating instructions. The drive system 28 receives the control signals from the controller 26 and actuates the plurality of valve actuators using a plurality of linear actuators. The drive system actuates the plurality of valve actuators in accordance with the control signals, thereby providing a valve configuration within the membrane valve cassette to enable the base unit 60 and the membrane valve cassette together to perform the identified bioprocessing operation.

FIG. 2B is a flow diagram showing operation of the control system shown in FIG. 2A. At 30, the control system receives an input via an input interface. The input to the control system may include one or more identifiers of membrane valve cassettes attached to the base unit 60, along with a desired bioprocessing operation.

At 32, a controller of the control system identifies, based on the received input, a bioprocessing operation from a predetermined set of bioprocessing operations. The bioprocessing operation may be identified based on the identifiers of the membrane valve cassettes and/or based on the desired bioprocessing operation received via the input interface.

At 34, the controller of the control system retrieves instructions for the identified bioprocessing operation from an operating instructions store that stores operating instructions for the predetermined set of bioprocessing operations.

At 36, the controller of the control system outputs signals to a drive system to control the drive system in accordance with the operating instructions retrieved from the operating instructions store.

FIG. 3 shows a valve actuation unit 100 that comprises a plurality of valve actuators in the form of an array of rods 102. As noted above, the valve actuation unit 100 may, for example, be mounted in a base unit 60 of a bioprocessing system 80 configured to perform a set of predetermined bioprocessing operations. However, the valve actuation unit 100 may alternatively be implemented in other liquid handling systems.

The array of rods 102 is provided on a surface 104 of the valve actuation unit 100. In the example shown in FIG. 3, the rods 102 are provided in a four by three array. Each of the array of rods 102 is movable between a first position in which the rod 102 is retracted (a retracted position), and a second position in which the rod 102 is engaged (an engaged position). In the engaged position, the rod 102 protrudes further from the surface 104 than in the retracted position.

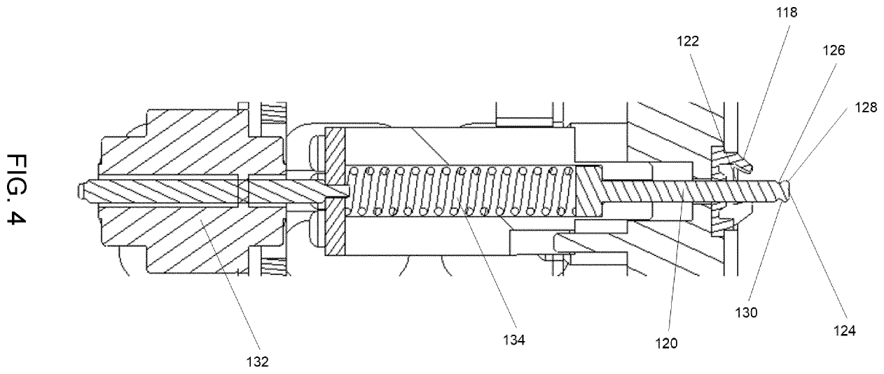

The rods 102 are driven by a drive system (not shown in FIG. 3) comprising a plurality of linear actuators, in which each rod 102 is independently mechanically actuated by a respective linear actuator. Specifically, each of the rods 102 is moved in a first direction (e.g. from the first position to the second position) and in a second direction (e.g. from the second position to the first position) by a corresponding linear actuator 132 (as best shown in FIG. 4). A controller (not shown) provides electrical control signals from the control system of the bioprocessing system 80 to the drive system in order to selectively control movement of each of the rods 102 between the first position and the second position in accordance with the valve configuration for the bioprocessing operation being performed.

The valve actuation unit 100 also includes a plurality of protrusions 106. Each protrusion 106 protrudes from the surface 104. In the example shown in FIG. 3, there are six protrusions. A first group 108 of three protrusions 106 is disposed on a first side of the array of rods 102. A second group 110 of three protrusions 106 is disposed on a second side of the array, the second side being opposite to the first side.

Each protrusion 106 includes a projection 112 at its distal end (i.e. the end of the protrusion 106 furthest from the surface 104). Each projection 112 protrudes from a face of the protrusion 106 that faces the array. As explained further below, the protrusions 106 are used to hold a membrane valve cassette in attachment with the valve actuation unit 100. Specifically, the membrane valve cassette is held in place by snap-fit attachment between opposing protrusions 106, with a portion of the membrane valve cassette being held between the surface 104 and the projection 112 when the membrane valve cassette is attached. The projections 112 include a bevelled surface 114 to allow for insertion of the membrane valve cassette between opposing protrusions 106.

FIG. 4 is a longitudinal sectional view through one of the rods 102 shown in FIG. 3. The rod 102 comprises a shaft 120 of circular cross-section, that extends through a hole 122 in the surface 104. Movement of the shaft 120 through the hole 122 is effected by the drive system. The valve actuation unit 100 includes a plurality of sealing portions 118, one of which is shown in FIG. 4. Each sealing portion 118 extends from the surface 104 and surrounds a corresponding shaft 120. The sealing portions 118 seal the membrane valve cassette when attached to the valve actuation unit 100 (for example, to provide IP55 rated sealing). The sealing portion 118 is formed of a resiliently deformable material which applies a force to the membrane valve cassette when attached to the valve actuation unit 100, which acts to push the membrane valve cassette away from the surface 104. The force applied by the sealing portion 118 helps to hold the membrane valve cassette in place on the valve actuation unit 100. The plurality of sealing portions 118 also ensure that there is a consistent distance between the membrane valve cassette and each rod 102, thereby ensuring that a consistent force is applied to close each valve.

The rod 102 also includes a head portion 124 at the end of the shaft 120. The head portion 124 is defined by a neck 126 of smaller diameter than the shaft 120.

The head portion 124 has a first rounded or bevelled edge 128 at its distal end (i.e. the end of the shaft 120 that protrudes from the surface 104). The head portion 124 is configured to fit within a receiving mechanism (e.g. a cavity) of a corresponding valve interface, as explained further below. The first rounded or bevelled edge 128 allows the head portion 124 to pass through a resiliently deformable collar of the valve interface during insertion of the head portion 124 into the cavity.

When the head portion 124 is fully inserted into the cavity, the resiliently deformable collar engages a corresponding portion (e.g. the neck 126) of the rod 102. The engagement between the resiliently deformable collar and the neck 126 provides a positive engagement between the rod 102 and the valve interface, which allows the rod 102 to actuate the valve interface by exerting a pushing force to move the valve interface in one direction and a pulling force to move the valve interface in an opposite direction.

To allow for removal of the head portion 124 from the cavity, the head portion 124 also includes a second rounded or bevelled edge 130 at a second end disposed opposite to the distal end of the head portion 124. The second rounded or bevelled edge 130 allows the head portion 124 to pass back through the deformable collar of the valve interface during removal of the head portion 124 from the cavity.

When the rod 102 is in the first position, a portion of the shaft 120 protrudes from the hole 122 so that the head portion 124 of the rod 102 can be inserted within the cavity of the valve interface.

Movement of the rod 102 is effected by the linear actuator 132. The linear actuator applies a force to a spring 134 mounted in series with the shaft 120. The spring 134 buffers the force applied by the linear actuator 132. To close a valve, a force is applied by the linear actuator 132 via the spring 134. This actuates the rod 102 to the engaged position to close the valve. An additional closing force can then be applied to the rod 102 via the linear actuator 132 and spring 134, so that the valve is not opened by the pressure of fluid flowing within the membrane valve cassette.

FIGS. 5A and 5B schematically illustrate a membrane valve cassette 200 comprising an array of valve interfaces 202. FIG. 5A shows an underside of the membrane valve cassette 200, showing a base 206 with a number of holes 208. FIG. 5B shows the underside of the membrane valve cassette 200 but with the base 206 removed.

The membrane valve cassette 200 provides a generic valve interface arrangement that may be implemented in a number of different cassettes that are tailored to bioprocessing operations. For example, the membrane valve cassette may be implemented as a configurable flow path cassette (e.g. as shown in FIG. 10), a flow path air trap cassette comprising an air trap (e.g. the air trap in FIG. 13), a flow path pressure monitoring cassette, which may be used in combination with a pressure sensing device (e.g. the pressure sensing device in FIG. 14), or a system valve cassette (e.g. as shown in FIGS. 11 and 12). In each case, the membrane valve cassette 200 comprises a housing 204. As best shown in FIG. 5A, the housing 204 has a base 206 that includes a number of holes 208. Each valve interface 202 (best shown in FIG. 5B) is disposed in one of the holes 208. In the example shown in FIGS. 5A and 5B, the valve interfaces 202 are provided in a four by three array, to correspond with the array of rods 102 shown in FIG. 3.

Returning to FIG. 5A, it can be seen that the housing 202 of the membrane valve cassette 200 comprises a first end wall 210 and a second end wall 212 opposite to the first end wall 210. The membrane valve cassette 200 also includes a first side port 214 in the first end wall 210, and a second side port 216 in the second end wall 212.

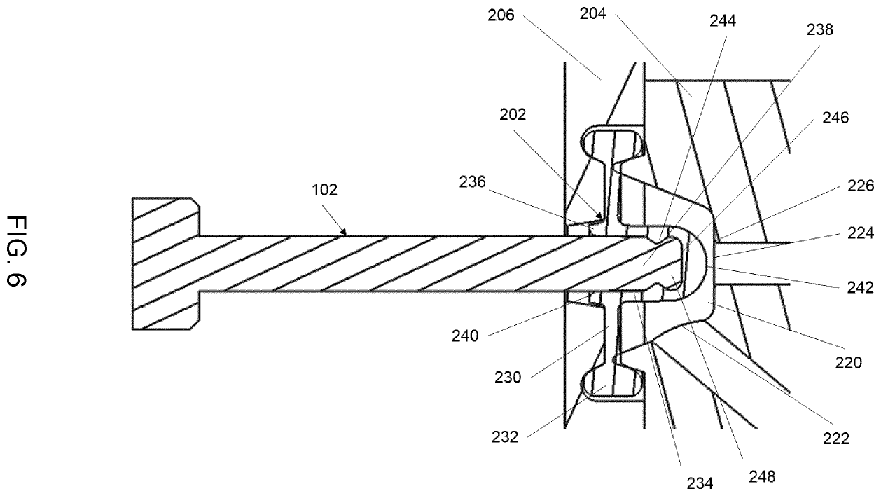

FIG. 6 is a cross-sectional view through one of the valve interfaces 202 shown in FIGS. 5A and 5B, showing a corresponding rod 102 in engagement with the valve interface 202. As shown in FIG. 6, the housing 204 comprises a chamber 220 accessible via a respective one of the holes 208 in the base 206 of the membrane valve cassette 200. The chamber 220 is also accessible via a first fluid port 222 in a side wall of the chamber 220 and a second fluid port 224 in a back wall of the chamber 220. For example, the first fluid port 222 may be a fluid entry port and the second fluid port 224 may be a fluid exit port (although the opposite configuration is also possible). The chamber 220 also includes a valve seat 226 coincident with the second fluid port 224.

The valve interface 202 comprises a resiliently deformable membrane 230 held in place within the housing 204. The resiliently deformable membrane 230 is provided in the form of a disc-shaped diaphragm that is anchored at its periphery 232 within the housing 204. The membrane 230 includes a valve body 234 located at the centre of the membrane 230. The valve body 234 is moveable within the chamber 220. The membrane 230 (including the valve body 234) may be formed of rubber having a Shore hardness of about 70.

An opening portion 236 of the valve body 234 is disposed within the respective hole 208 through which the chamber 220 is accessible. The valve body 234 includes a receiving mechanism in the form of an internal cavity 238 accessible via the opening portion 236. The receiving mechanism is configured to releasably engage a respective rod 102 when the membrane valve cassette 200 is attached to the valve actuation unit 100. The opening portion 236 includes a bevelled edge 240 to allow for insertion of the head portion 124 of the rod 102 into the internal cavity 238.

The valve body 234 has an elongate shape with a rounded portion 242 at the opposite end to the opening portion 236. The rounded portion 242 engages with the valve seat 226 when the valve interface 202 is actuated (specifically, when a corresponding rod 102 is moved to the second position). When the rounded portion 242 of the valve body 234 engages with the valve seat 226, the flow of fluid between the first fluid port 222 and the second fluid port 224 is prevented. Likewise, when the rounded portion 242 of the valve body 234 is brought out of engagement with the valve seat 226 (e.g. when the rod 102 is moved from the second position towards the first position), fluid is able to flow between the first fluid port 222 and the second fluid port 224.

The valve body 234 also includes a circular ridge in the form of a resiliently deformable collar 244 disposed within the cavity 238. The resiliently deformable collar 244 extends into the interior of the cavity 238. The resiliently deformable collar 244 is configured to deform in order to allow the head portion 124 of the rod 102 to pass through. However, the resiliently deformable collar 244 also provides some resistance to removal of the head portion 124, in order to allow the rounded portion 242 to be pulled out of contact with the valve seat 226 by retracting the head portion 124 from the second position to the first position.

The distance between an interior end 246 of the cavity 238 and the resiliently deformable collar 244 is substantially the same as the length of the head portion 124, in order to allow the head portion 124 to engage both the interior end 246 of the cavity 238 and a surface of the flexible collar 244 that opposes the back wall. This means that the head portion 124 pushes on the interior end 246 when the rod 102 is moved from the first position to the second position. Likewise, the head portion 124 pulls on the surface of the flexible collar 244 opposite to the interior end 246 when the rod 102 is moved from the second position to the first position.

The resiliently deformable collar 244 engages with a corresponding portion of the corresponding rod 102 (e.g. the neck 126 of the corresponding rod 102), providing a positive engagement between the rod 102 and the valve interface 202. As noted above, this allows the rod 102 to actuate the valve interface 202 by exerting a pushing force to move the valve interface 202 in one direction and a pulling force to move the valve interface 202 in an opposite direction. The positive engagement between the rod 102 and the valve interface 202 allows the valve interface 202 to be pulled away from the second position. This improves the reliability of the valve interface 202 by avoiding the valve interface 202 becoming stuck in the second position. Exerting a pulling force to move the valve interface 202 also improves the speed of operation of the valve interface 202.

The cavity 238 includes an internal chamber 248 defined between the interior end 246 of the cavity 238 and the resiliently deformable collar 244. As shown in FIG. 6, the volume of the chamber 248 is substantially less than the volume of the cavity 238. For example, the volume of the chamber 248 may be less than half of the volume of the cavity 238. Using this volume of chamber 248 reduces or eliminates the amount of air that is trapped within the cavity when the head portion 124 is inserted into the cavity 238.

In other words, the distance between the interior end 246 of the cavity 238 and the resiliently deformable collar 244 is less than half of the distance between the interior end 246 of the cavity 238 and the bevelled edge 240 (in other words, less than half of the depth of the cavity 238, where the depth is defined by the interior end 246 of the cavity 238). Using this distance prevents air from being trapped within the cavity 238 when the head portion 124 is inserted through the resiliently deformable collar 244.

In particular, any air trapped within the cavity 238 is released by applying a force to push the rod 102 against the valve seat 226, which pressurises the air within the cavity 238. The pressurised air deforms the collar 244, which releases the air from within the cavity 238, resulting in the air being expelled towards the base of the rod 102.

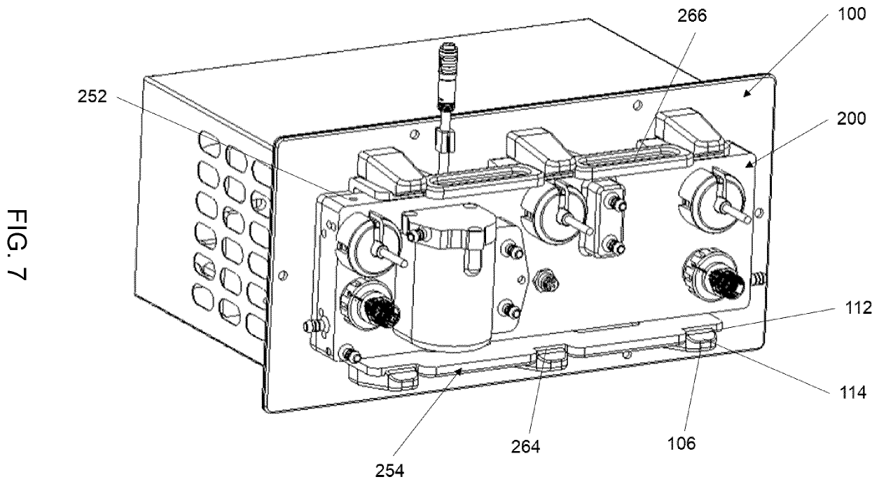

FIGS. 7-9 show the features of the membrane valve cassette 200 that provide for attachment of the membrane valve cassette 200 to the valve actuation unit 100. Although FIGS. 7-9 show a membrane valve cassette in the form of a system valve cassette (described in more detail with reference to FIGS. 11-14), the features that provide for attachment to the valve actuation unit 100 may also be incorporated into the reconfigurable flow path cassette 300 described with reference to FIGS. 10A-10C, as seen in particular in FIG. 10B.

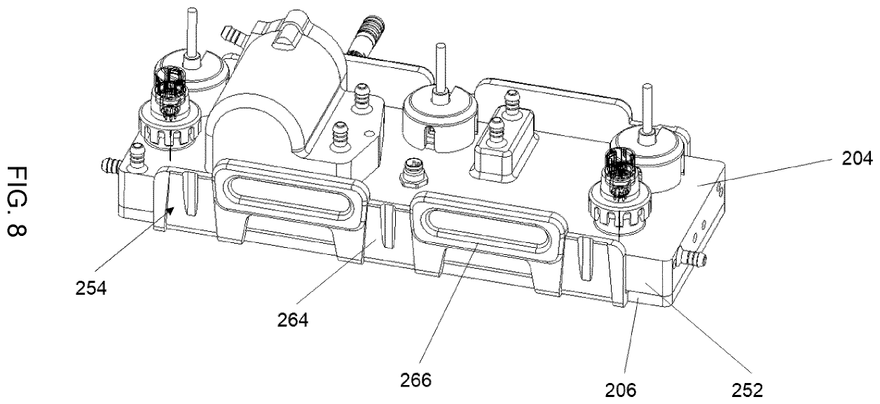

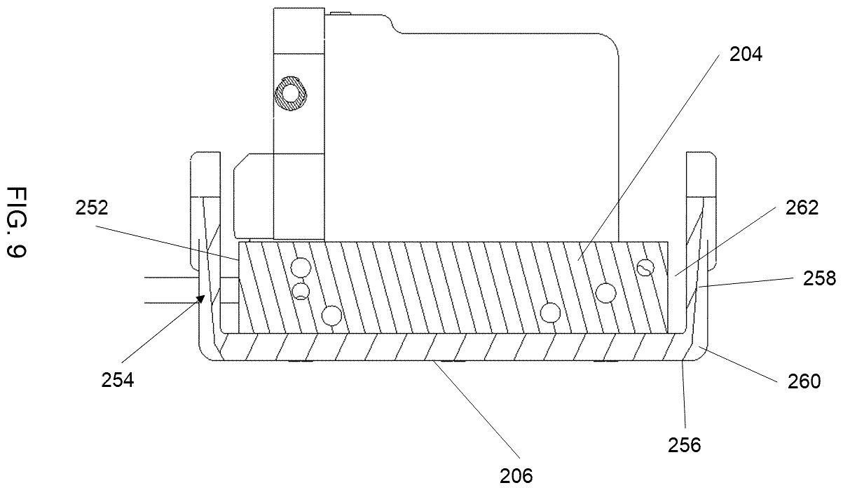

FIG. 7 shows the membrane valve cassette 200 in snap fit attachment with the valve actuation unit 100. As best shown in FIGS. 8-9, the membrane valve cassette 200 includes an integral attachment mechanism in the form of L-shaped winged sections 254 extending from side walls 252 of the housing 204. Each of the winged sections 254 includes a plurality of grooved sections 264 (as best shown in FIG. 8). The grooved sections 264 allow the membrane valve cassette 200 to be snap fit between the protrusions 106 of the valve actuation unit 100, as shown in FIG. 7.

The grooved sections 264 also help to align the membrane valve cassette 200 in the correct horizontal orientation. This is because the membrane valve cassette 200 is attached by aligning the protrusions 106 with the grooved sections 264.

In addition, as the membrane valve cassette 200 is pushed towards the surface 104, the head portion 124 of each rod 102 extends into the cavity 238 of a corresponding valve interface 202 and subsequently through the resiliently deformable collar 244, to fit within the chamber 248. Once the head portion 124 is held in place within the chamber 248 by the flexible collar 244, the rod 102 can be moved between the first and second positions in order to bring the rounded portion 242 of each valve body 234 into and out of engagement with the valve seat 226.

In order to ensure that the rods 102 all engage with the valve interfaces 202 at the same time when the membrane valve cassette 200 is pushed towards the surface 104, the height of the grooved sections 264 is the same as the distance between the surface 104 and the base of each projection 112.

The distance between the winged sections 254 may be smaller than a human handspan, so that an operator can use their hands to squeeze the winged sections 254 on opposing sides of the housing 204 towards each other in order to bring the grooved sections 264 out of engagement with the protrusions 106, thereby allowing the membrane valve cassette 200 to be removed from the valve actuation unit 100. For example, the distance between the winged sections may be between 80 mm and 150 mm. There may be a corresponding distance between the first group 108 and second group 110 of protrusions on the surface 104 of the valve actuation unit 100.

FIG. 8 shows that each winged section 254 extends along the length of a side wall 252 of the housing 204. FIG. 8 also shows the grooved sections 264 that allow the winged sections 254 to be snap fit within the protrusions 106 of the valve actuation unit 100.

The extension of the winged sections 254 from the housing 204 of the membrane valve cassette 200 is shown in more detail in FIG. 9, which is a cross section through the membrane valve cassette 200 shown in FIGS. 7 and 8. As explained above, the membrane valve cassette 200 shown in FIGS. 7-9 is in the form of a system valve cassette. An air trap of the system valve cassette can be seen in FIG. 9. The air trap is described in more detail with reference to FIGS. 11-13.

FIG. 9 shows the housing 204 of the membrane valve cassette 200 comprising the base 206 and side walls 252 that are perpendicular to the base 206. The winged sections 254 that extend from each side wall 252 are integral with the base 206. The winged sections 254 and base 206 may be formed of a different material to the housing 204, because the winged sections 254 and base 206 are not wetted parts of the membrane valve cassette 200 (i.e. they do not come into contact with the fluid). Suitable materials for the housing 204 include, for example, polypropylene, polymethylpentene (also known as TPX™), and cyclic olefin copolymer (COC).

As seen in FIG. 9, each winged section 254 comprises a first portion 256 that is parallel to the base 206, a second portion 258 that is parallel to the side wall 252, and a curved portion 260 joining the first portion 256 to the second portion 258.

The curved portion 260 allows the winged section 254 to function as a resiliently deformable hinge. Specifically, when pressure is applied to one side of the second portion 258, the second portion 258 hinges about the curved portion 260. The first portion 256 extending parallel to the base 206 provides a gap 262 between the side wall 252 and the second portion 258, allowing the second portion 258 to be pushed inwards toward the side wall 252.

As best shown in FIG. 8, the plurality of grooved sections 264 are formed in the second portion 258 and the curved portion 260. In each grooved section 264, the second portion 258 and the curved portion 260 have a reduced thickness, thereby forming a groove in the winged section 254. In addition, the length of the second portion 258 (i.e. the distance that the second portion 258 extends from the curved portion 260) is reduced in each grooved section 264.

FIG. 8 also shows that the second portion 258 of each winged section 254 includes ridges 266 extending longitudinally between the grooved sections 264. The ridges 266 protrude outwardly from each second portion 258. The ridges 266 allow an operator to push the second portions 258 of the winged sections 254 inwards in order to release the membrane valve cassette 200 from being held by the protrusions 106.

In order to attach the membrane valve cassette 200 to the valve actuation unit 100, the grooved sections 264 are initially aligned with the protrusions 106. As the membrane valve cassette 200 is pushed towards the surface 104, the projections 112 push the second portions 258 of the winged sections 254 inwards, before snapping over the ends of the grooved sections 264, as shown in FIG. 7.

The array of rods 102 and corresponding array of valve interfaces 202 allow a range of different membrane valve cassettes 200 to be removably attached to the valve actuation unit 100. This allows a wide variety of different flow paths to be achieved, using the same standard interface, where the array of rods 102 control the flow of fluid through conduits in each membrane valve cassette 200. As a result, the bioprocessing system 80 can be reconfigured to carry out a range of different bioprocessing operations (e.g. chromatography, mixing, filtration, etc.)

Two examples of membrane valve cassettes 200 that can be used with the valve actuation unit 100 are shown in FIGS. 10A to 10C (configurable flow path cassette 300) and FIG. 11 (system valve cassette 400). It will be appreciated, however, that additional flow paths and components can be integrated using the same interface.

FIG. 10A is a schematic diagram of a configurable flow path cassette 300, which is a first example of a membrane valve cassette that can be used with the valve actuation unit 100. The configurable flow path cassette 300 is shown in isometric view in FIG. 10B. The configurable flow path cassette 300 comprises all of the features of the generic membrane valve cassette 200 described above.

In addition, the configurable flow path cassette 300 has a housing 302 that includes all features of the housing 204 of the generic membrane valve cassette 200, in addition to a front wall 308 opposite to the base 206. The configurable flow path cassette 300 further includes a plurality of fluid ports 310 disposed on the front wall 308 of the housing 302.

Together, the front wall 308, base 206, side walls 252, first end wall 210 and second end wall 212 define an interior volume of the housing 302. The configurable flow path cassette 300 includes a plurality of conduits 312 provided within the interior volume of the housing 302. The plurality of conduits 312 connect the fluid ports 310 to the first side port 214 and second side port 216. This means that the first side port 214 and second side port 216 are used for the inlet and/or outlet of fluids to/from the conduits 312. Fluid flow through the conduits 312 (and consequently through the ports 214, 216, 310) is controlled by actuation of the valve interfaces 202 by the array of rods 102 on the valve actuation unit 100.

In the example shown in FIG. 10A, the valve interfaces 202 are provided in the form of a four by three array (i.e. there are twelve valve interfaces 202). In addition, there are ten fluid ports 310 in the example of FIG. 10A. Fluid flow through each of the ten fluid ports 310 is controlled by a corresponding valve interface 202. The ten fluid ports 310 are divided into a first group 314 of seven ports and a second group 316 of three ports (as best shown in FIG. 10B). Returning to FIG. 10A, it can be seen that the first group 314 of ports is in selective fluidic communication with the first side port 214 (where selective fluid communication means that flow through the individual ports 310 in the first group 314 of ports is controlled using the corresponding valve interfaces 202).

The remaining two valve interfaces 202 (indicated in FIG. 10A as valve interfaces 304 and 306) are used to control the fluidic communication between the second group of ports 316 and the first side port 214, and to control the fluidic communication between the second group of ports 216 and the second side port 216. Specifically, the valve interface 304 is used to control whether or not the second group 316 of ports is selectively fluidically connected to the first side port 214 (where selectively fluidically connected means that even if the second group 316 of ports is fluidically connected to the first side ports 214, flow through the individual ports 310 in the second group of ports 314 is selectively controlled using the corresponding valve interfaces 202). Likewise, the valve interface 306 is used to control whether or not the second group 316 of ports is selectively fluidically connected to the second side port 216.

In this way, the configurable flow path cassette 300 provides the following fluidic communication configurations: (i) the first group 314 and second group 316 of ports (i.e. all ten ports 310) in selective fluidic communication with the first side port 214, with no ports in fluidic communication with the second side port 216; (ii) the first group 314 of ports (i.e. seven ports) in selective fluidic communication with the first side port 214, with the second group 316 of ports (i.e. three ports) in selective fluidic communication with the second side port 216; (iii) the first group 314 of seven ports in selective fluidic communication with the first side port 214, with the second group 316 of three ports in selective fluidic communication with neither of the side ports 214, 216; or (iv) the first group 314 of seven ports in selective fluidic communication with the first side port 214, with the second group 316 of three ports in selective fluidic communication with both side ports 214, 216.

As one example, the configurable flow path cassette 300 may be used to control the flow of fluids to two pumps: pump A and pump B (such as the pumps 84 shown in FIG. 1B). Returning to FIG. 10A, the first group 314 of ports is selectively fluidically connected to the first side port 214. The first side port 214 is, in turn, fluidically connected to pump A. This means that between zero and seven of the inlets connected to the first group 314 of ports can be brought into fluidic communication with pump A by controlling the valve interfaces 202 corresponding to each of the first group 314. An additional three ports (i.e. the second group 316 of ports) can be brought into fluidic communication with pump A using the valve interface 304, allowing between zero and ten inlets to be brought into fluidic communication with pump A.

Alternatively, the second group 316 of ports may be is selectively fluidically connected to the second side port 216, which is, in turn, fluidically connected to pump B, using the valve interface 306. This means that between zero and three of the inlets connected to the second group 316 of ports can be brought into fluidic communication with pump B by controlling the valve interfaces 202 corresponding to each of the second group 316.

As a further alternative, the second group 316 of ports may be selectively fluidically connected to the first side port 214 and the second side port 216, meaning that the inlets connected to the second group 316 of ports can be brought into fluidic communication with pump A and pump B. In this case, the split of flow from the second group 316 of ports between pump A and pump B would be determined by the speeds of the pumps.

It will be appreciated that the above example of using the configurable flow path cassette 300 to provide a selectable number of inlet flows is an example only, and that the configurable flow path cassette 300 may alternatively be used to route fluid outlets from a particular process (e.g. as described above with reference to FIG. 1B), or for both inlet and outlet.

An example of using the configurable flow path cassette 300 to route fluid outlets is shown in FIG. 10C. In the example shown in FIG. 10C, the first side port 214 is in selective fluidic communication with the first group 314 of ports (FIG. 10 shows all seven of the first group 314 of outlets in fluidic communication with the first side port 214). In addition, the second side port 216 is in selective fluidic communication with the second group 316 of ports. In the example shown in FIG. 10C, two of the second group 316 of ports are plugged using the valve interfaces 202 (i.e. so that there is no flow through them), while the third port is in fluidic communication with the second side port 216. In this example, the air outlet from the air trap is routed to the second side port 216 and subsequently to the open port of the second group 316 of ports (i.e. as with the example shown in FIGS. 1B and 1C). The air flow from the open port may then be routed through an air filter (e.g. as shown in FIG. 1C).

FIG. 11 is a schematic diagram of a system valve cassette 400, which is a second example of a membrane valve cassette that can be used with the bioprocessing system 80. The system valve cassette 400 comprises all of the features of the generic membrane valve cassette 200 described above. In addition, the system valve cassette 400 has a housing 402 that includes all features of the housing 204 of the generic membrane valve cassette 200.

The system valve cassette 400 comprises a front face 404 having a first pair 408 of fluid ports and a second pair 410 of fluid ports disposed therein. Together, the front face 404, base 206, side walls 252, first end wall 210 and second end wall 212 define an interior volume of the housing 402. As best illustrated in FIG. 12, the flow processing module 400 also includes a plurality of conduits 490 provided within the interior volume of the housing 402.

FIG. 12 shows that the plurality of conduits 490 fluidically connect the following components of the flow processing module 400: an air trap 406, the first pair 408 of ports, and the second pair 410 of ports. The valve interfaces 202 control the fluidic connection to these components. That is, the valve interfaces 202 control: (i) whether fluid can flow through the air trap 406 shown in FIG. 11 or whether the fluid bypasses the air trap 406; (ii) whether fluid can flow through the first pair 408 of ports or whether the fluid bypasses the first pair 408 of ports; and (iii) whether fluid can flow through the second pair 410 of ports or whether the fluid bypasses the second pair 410 of ports.

FIG. 12 schematically shows the locations of the first pair 408 of ports and the second pair 410 of ports, along with an inlet 426 and outlet 428 to the air trap 406, and the plurality of conduits 490 that fluidically connect the components of the system valve cassette 400. In addition, FIG. 12 shows the locations of the pressure sending devices 412, conductivity sensing devices 414 and the air detection sensor 416. FIG. 12 also shows that one of the valve interfaces 202 can be used to provide a fluidic connection to a waste conduit 492.

The air trap 406 is configured to trap any air flowing through a conduit 490. The housing 402 also includes two sensors, such as level sensors 420, 422 (shown in FIG. 13), integrated within the housing 402. The two level sensors 420, 422 monitor the level of fluid within the air trap 406. Specifically, as shown in FIG. 13, a first level sensor 420 monitors whether the level of fluid falls below a first level, and a second level sensor 422 monitors whether the level of fluid exceeds a second level that is higher than the first level. If the level of fluid falls below the first level, then an air outlet 424 (shown in FIG. 11) is opened in order to release air from the air trap 406 and thereby increase the level of fluid. The air outlet 424 may further include a filter to filter the air released from the air trap 406. This avoids contamination of the ambient air by any substances present in the air released from the air trap 406. If the level of fluid exceeds the second level, then the air outlet 424 is closed to prevent air from being released from the air trap 406.

As shown schematically in FIG. 12, the air trap 406 also includes an inlet 426 in fluidic communication with one of the conduits 490, and an outlet 428 in fluidic communication with another one of the conduits 490.

In the example described above with reference to FIG. 11 and FIG. 12, the air trap 406 is part of a system valve cassette 400 that is used with the bioprocessing system 80. However, the air trap 406 may be implemented in conjunction with the generic structure of the membrane valve cassette 200 in order to provide a flow path air trap cassette for a bioprocessing system. The flow path air trap cassette may include one or more conduits and an air trap having all of the features of the air trap 406 shown in FIG. 13. The flow path air trap cassette may therefore be configured to capture any air present in fluid flowing through one or more of the conduits. The flow path air trap cassette may also include any of the additional components of the system valve cassette 400 described with reference to FIG. 11 and FIG. 12, and/or the configurable ports of the configurable flow path cassette described with reference to FIGS. 10A to 10C.

Returning to FIG. 11, the first pair 408 of ports includes an outlet from the system valve cassette 400 and an inlet back to the system valve cassette 400. The inlet and outlet of the first pair 408 of ports may be fluidically connected to a filter (e.g. a coarse filter) configured to remove aggregates from the fluid flowing through it. In the example shown in FIG. 11, the filter is located external to the system valve cassette 400 (i.e. it is not a component of the system valve cassette 400). The filter may be part of a flow handling kit that includes the system valve cassette 400 and, optionally, tubing to connect the system valve cassette 400 and the filter.

The second pair 410 of ports also includes an outlet from the system valve cassette 400 and an inlet back to the system valve cassette 400. The inlet and outlet of the second 410 pair of ports may be fluidically connected to an external chromatography column (e.g. the chromatography column 70 shown in FIGS. 1A and 1B). The chromatography column is configured to carry out a chromatography process (e.g. a fluid chromatography process such as a liquid chromatography process) on the fluid flowing through the column. Returning to FIG. 11, the valve interfaces 202 control the flow of fluid through the second pair 410 of ports, thereby controlling the direction of fluid flow through the column. For example, the valve interfaces 202 may be controlled to allow fluid to flow through the chromatography column in a “top down” arrangement. Following the top down chromatography process, the chromatography column may be flushed in order to remove any accumulated biomolecules within the column. This may be achieved by carrying out a reverse flush of the chromatography column, by controlling the valve interfaces 202 in order to switch the inlet and outlet of the second pair 410 of ports, and subsequently running an elution reagent through the column.

The system valve cassette 400 further includes an attachment mechanism in the form of hollow projections 436 (as best shown in FIG. 14) that allow a plurality of pressure sensing devices 412 to be fitted to the front face 404 of the housing 402. Returning to FIG. 11, the plurality of pressure sensing devices 412 includes a first pressure sensing device configured to detect the pressure of fluid in a conduit connected to the outlet of the second pair 410 of ports and a second pressure sensing device configured to detect the pressure of fluid in a conduit connected to the inlet of the second pair 410 of ports. This allows the pressure at the inlet to the chromatography column and the outlet from the chromatography column to be measured, thereby allowing the difference in pressure across the chromatography column to be measured. Certain chromatography processes require a particular level of pressure for the process flow through the chromatography column. Measuring the pressure across the chromatography column using the pressure sensing devices 412 therefore allows an operator to determine whether there is sufficient pressure across the chromatography column for a particular process.

The plurality of pressure sensing devices 412 also includes a third pressure sensing device configured to detect the pressure of fluid in a conduit connected to the first side port 214. This allows the pressure at the inlet to the filter to be measured, which allows an operator to determine whether anything is blocking the filter (which would lead to increased pressure at the inlet).

Each of the pressure sensing devices 412 has the same construction, which is shown in more detail in FIG. 14. In the example shown in FIG. 14, each pressure sensing device 412 includes a PendoTECH single-use pressure sensor available from PendoTECH of Princeton, New Jersey, U.S. The pressure sensor is mounted within a cylindrical casing 430 that provides for snap-fit attachment of the pressure sensing device 412 to a corresponding attachment mechanism (e.g. hollow cylindrical projection 436) provided on the front face 404 of the housing 402. Each cylindrical projection 436 has an outwardly projecting lip 438. Each casing 430 includes a plurality of projections 432 that extend inwardly from an interior surface of the casing 430. When the casing 430 is pushed onto the cylindrical projection 436, the plurality of projections 432 snap over the lip 438 to provide a snap fit attachment between the pressure sensing device 412 and the cylindrical projection 430. To facilitate the snap fit attachment, the lip 438 comprises an angled surface 440 that pushes the projections 432 outwards when the casing 430 is pushed onto the cylindrical projection 436. In addition, the casing 430 includes cutouts 434 in the cylindrical surface to allow portions of the cylindrical surface adjacent the cutouts 434 to flex outwardly when the casing 430 is pushed onto the cylindrical projection 436.

The pressure sensing device 412 also includes a substantially cylindrical support 442 disposed within the casing 430. The support 442 is sized to fit within the cylindrical projection 436, meaning that there is an annular gap between the support 442 and the casing 430. Two O-ring seals 444 are disposed on the cylindrical surface of the support 442, to allow the pressure sensing device 412 to be held in place within the cylindrical projection 436.

A sensing area 446 is disposed on a portion of the flat end of the cylindrical support 442. The sensing area 446 is configured to detect a pressure of a fluid in an adjacent conduit 490 accessible via a hole in a base 452 of the cylindrical projection 436. In addition to the O-ring seals 444 provided on the cylindrical surface of the support 442, a third O-ring seal 448 is provided at the base 452 of the cylindrical projection 436. This means that the third O-ring seal 448 contacts the flat end of the cylindrical support 442 when it is pushed into the cylindrical projection 436. Specifically, the third O-ring seal 448 surrounds the sensing area 446 on the flat end of the cylindrical support 442 when the pressure sensing device 412 is pushed into the cylindrical projection 436. The third O-ring seal 448 prevents leakage of fluid that comes into contact with the sensing area 446 (for example, leakage of fluid into the cylindrical projection 436. A fourth O-ring seal 450 of larger diameter than the third O-ring seal 448 is also provided at the base 452 of the cylindrical projection 436. The fourth O-ring seal 450 contacts the flat end of the cylindrical support 442 (specifically, the rim of the cylindrical support 442) when it is pushed into the cylindrical projection 436. The fourth O-ring seal 450 is provided to stabilise the pressure sensing device 412 in order to prevent the pressure sensing device 412 from tilting within the cylindrical projection 436. Stabilising the pressure sensing device 412 ensures that the seal provided by the third O-ring seal 448 is not compromised by tilting of the pressure sensing device 412 within the cylindrical projection 436.

The snap-fit attachment between the casing 430 and the cylindrical projection 436 (specifically, between the projections 432 and the lip 438) results in the application of pressure to the third and fourth O-ring seals 448, 450. This allows the pressure sensing device 412 to be balanced (i.e. not tilted) within the cylindrical projection 436, and prevents leakage of fluid through the seals 448, 450.

In the example described above with reference to FIG. 11 and FIG. 12, the projections 436 are part of a system valve cassette 400 that is used with the bioprocessing system 80. However, the projections 436 for receiving the pressure sensing devices 412 may be implemented in conjunction with the generic structure of the membrane valve cassette 200 in order to provide a flow path pressure monitoring cassette for a bioprocessing system. The flow path pressure monitoring cassette may include one or more conduits and one or more projections having all of the features of the projections 436 shown in FIG. 14. The flow path pressure monitoring cassette may therefore be configured to provide an attachment mechanism (e.g. in the form of a projection 436) configured to engage a pressure sensing device 412. The flow path pressure monitoring cassette and the pressure sensing device 412 may form part of a flow path pressure monitoring kit. When the pressure sensing device 412 is engaged with the attachment mechanism, a seal may contact the flat end at the distal end of the pressure sensing device 412. The pressure sensing device 412 may be configured to measure a pressure of fluid flowing through one or more of the conduits. The flow path pressure monitoring cassette may also include any of the additional components of the system valve cassette 400 described with reference to FIG. 11 and FIG. 12, and/or the configurable ports of the configurable flow path cassette described with reference to FIGS. 10A to 10C.

Returning back to FIG. 11, it can be seen that the system valve cassette 400 further includes a piezoelectric air detection sensor 416. The piezoelectric air detection sensor 416 is configured to detect the presence of air within one or more of the conduits 490 of the system valve cassette 400.

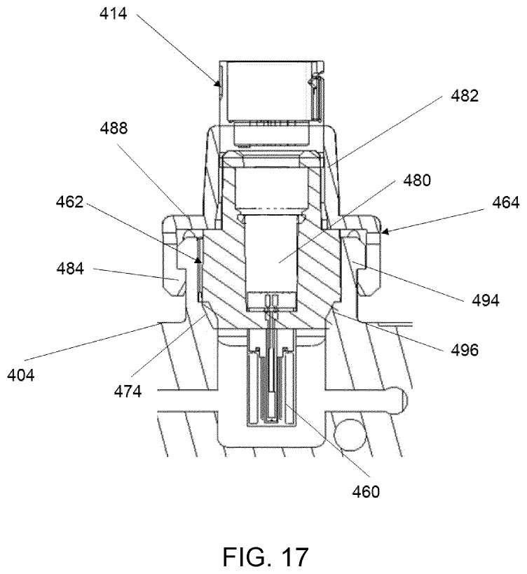

The system valve cassette 400 further includes hollow projections (shown in FIG. 17) that allow a plurality of conductivity sensing devices 414 (e.g. two conductivity sensing devices 414 as shown in FIG. 11) to be fitted to the system valve cassette 400. A first one of the conductivity sensing devices 414 is configured to detect a salt level in fluids flowing through a conduit 490 in fluidic communication with the first side port 214, while a second one of the conductivity sensing devices 414 is configured to detect a salt level in fluids flowing through a conduit 490 in fluidic communication with the second side port 216. The construction of each conductivity sensing device 414 is shown in further detail in FIG. 15.

As shown in FIG. 15, the conductivity sensing device 414 includes a conductivity sensor 460, a sensor holder 480 in which the conductivity sensor 460 is mounted, a sealing portion 462, and a casing in the form of a snap nut 464. The components of the conductivity sensing device 414 are shown in exploded view in FIG. 15 with the assembled conductivity sensing device 414 shown in FIG. 16. The sealing portion 462 is formed of an elastomeric material such as a thermoplastic elastomer. Returning to FIG. 15, it can be seen that the sealing portion 462 has a substantially cylindrical portion 466 with a flat face 468 (shown in FIG. 16) at a first end 470 (i.e. a distal end of the sealing portion 462). The face 468 includes an opening in the form of a slit 472. The slit 472 is sized to provide an interference fit for the conductivity sensor 460 when the conductivity sensor 460 is pushed through the slit 472. Returning to FIG. 15, the first end 470 further comprises a circular bevelled edge 474 around the face 468. The sealing portion has an opening 476 at a second end 478 opposite the first end 470. The opening 476 is larger than the slit 470 and is configured to receive the sensor holder 480 in which the conductivity sensor 460 is mounted. The sensor holder 480 provides an attachment point for a cable connector (not shown).

The snap nut 464 includes a cylindrical housing 482 that is configured to fit over the substantially cylindrical portion 466 of the sealing portion 462. The snap nut 464 (or casing) further includes attachment means in the form of a plurality of teeth 484, each projecting radially inwardly from a corresponding finger 486 that extends from a flange 488 located at one end of the cylindrical housing 482. The teeth 484 provide for snap-fit attachment of the snap nut 464 to a cylindrical projection provided on the front face 404 of the housing 402 of the system valve cassette 400 (as shown in FIG. 17), wherein the cylindrical projection has an outwardly extending lip that the teeth 484 snap over when the snap nut 464 is pushed onto the cylindrical projection. The cylindrical projections to which the snap nut 464 attaches are similar in construction to the cylindrical projections to which the pressure sensing devices 412 attach.

As shown in FIG. 17, the system valve cassette 400 includes hollow cylindrical projections 494 that allow a plurality of conductivity sensing devices 414 to be fitted to the front face 404 of the housing 402. Each cylindrical projection 494 includes an outwardly projecting lip. The plurality of teeth 484 snap over the lip of the cylindrical projection to provide a snap fit attachment between the conductivity sensing device 414 and the cylindrical projection 494. An annular angled surface 496 is provided within the cylindrical projection 494 at the base of the cylindrical projection 494. The annular angled surface 496 applies a force to the circular bevelled edge 474 of the conductivity sensing device 414, which is translated by the sealing portion 462 so that a force is applied to the conductivity sensor 460. The force applied to the conductivity sensor 460 by the sealing portion 462 means that the sealing portion 462 forms a seal around the conductivity sensor 460.

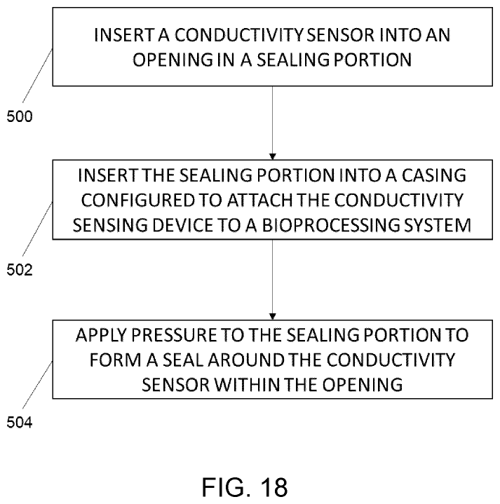

FIG. 18 is a flowchart of a method of manufacturing a conductivity sensing device. The method comprises, at 500, inserting a conductivity sensor 460 into an opening in a sealing portion 462. Specifically, the conductivity sensor 460 is inserted into a slit 472 provided in a face 468 at a first end 470 of a substantially cylindrical portion 466 of a sealing portion 462 formed of an elastomeric material. The slit 472 provides an interference fit for the conductivity sensor 460.

At 502, the method further comprises inserting the sealing portion 462 into a casing configured to attach the conductivity sensing device to a bioprocessing system (e.g. to a membrane valve cassette, such as the system valve cassette 400, configured for use with the bioprocessing system). Specifically, a substantially cylindrical portion 466 of the sealing portion 462 is inserted into a cylindrical housing 482 of a casing in the form of a snap nut 464. The snap nut 464 includes attachment means in the form of a plurality of teeth 484 that provide for snap-fit attachment of the snap nut 464 to the bioprocessing system (e.g. to a cylindrical projection on the housing 402 of the system valve cassette 400).

At 504, the method further comprises applying pressure to the sealing portion 462 to form a seal around the conductivity sensor 460 within the opening. Specifically, pressure may be applied to a bevelled edge 474 around the face 468 of the sealing portion 462 to apply pressure to the conductivity sensor 460 positioned within the slit 470. The pressure may be applied by snap-fit attachment of the snap nut 464 to the bioprocessing system (e.g. to the cylindrical projection 494 on the housing 402 of the system valve cassette 400). The snap-fit attachment may, for example, be provided by (i) a plurality of teeth 484 projecting radially inwardly from respective fingers 486 extending from a flange 488 located at one end of the housing 482 of the snap nut 464; and (ii) an outwardly extending lip on the cylindrical projection 494.

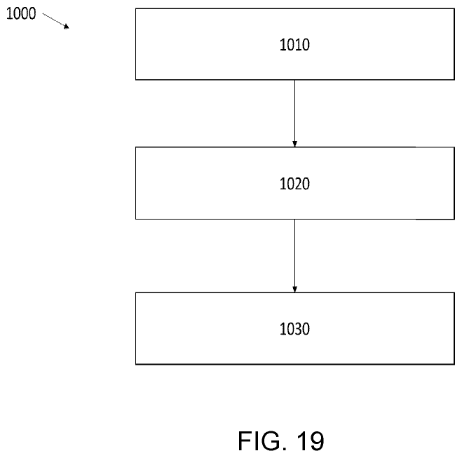

FIG. 19 shows a method 1000 for reconfiguring a bioprocessing system in accordance with various examples of the present disclosure. Such a method 1000 may be implemented by a control system used with the bioprocessing system (e.g. the control system 20 shown in FIG. 2A).

The method 1000 comprises a first step 1010 of replacing a first flow path kit 96 in a bioprocessing system 80 with a second flow path kit 96 for performing a bioprocessing operation that is different from that associated with said first flow path kit 96. For example, a first flow path kit 96 may be used to provide a mixing operation of various fluid components that are subsequently intended to be used in a chromatography separation process. A second flow path kit 96 may then be used to perform the chromatography operation using the mixed fluid components.

The method 1000 and its first step 1010 may comprise the fitting and configuration of membrane valve cassettes (and may further comprise a change in configuration or orientation of membrane valve cassettes and/or valve actuation units within the base unit 60).

The method 1000 comprises a second step 1020 of identifying a model and/or type of the second flow path kit 96. This step 1020 may include providing a graphical user interface (GUI) on a screen of a base unit 60. Such a screen may be touch sensitive and allow a user to identify the type and/or model of the second flow path kit 96. Such an identification allows the base unit 60 to program the control system therein to control, inter alia, the valve actuators (e.g. rods 102) in order to provide appropriate fluid flow control within the second flow path kit 96 to achieve a desired bioprocessing operation.

Whilst manual identification is possible, in preferred implementations automatic identification of a membrane valve cassette is provided. Such a membrane valve cassette can be automatically identified when it is provided for use in a bioprocessing system 80. The membrane valve cassette, or any other part of the flow path kit 96, may thus be provided with a radio-frequency identification (RFID) tag, a 2D bar code, a near-field communication tag, an optically readable tag etc. that, for example, encodes a unique identifier (UID). The base unit 60 (e.g. at each of the valve actuation units 100, membrane valve cassettes thereof) may thus be configured to read the UID from the membrane valve cassette as it is installed for use, for example.

The base unit 60 may be connected to a communications network. For example, a secure internet link may be provided that connects the base unit 60 to a secure remotely hosted central database. A record in the central database may thus be created for each base unit 60 and/or each flow path kit that is sold/provided. These records can be used to identify if a particular flow path kit 96 presented to a particular base unit 60 is compatible therewith, is a genuine part, has already been used (and thus possibly faked), has been sterilised correctly, is subject to a product recall, is out of date, etc. Use of a specific flow path kit 96 with a base unit 60 may thus be inhibited as appropriate thereby e.g. so as to prevent any contaminated bioproducts being produced.

In various embodiments, the base unit 60 may also provide process data and/or control system data to the central database to enable data analytics to be undertaken to provide diagnostics etc. for the base unit 60 itself and/or any flow path kits used therewith. Faults with a base unit 60 may also be reported, e.g. thereby enabling servicing personnel to be alerted.

The method 1000 also comprises a third step 1030 of reconfiguring the bioprocessing system 80 based upon the model/type identification information of the second flow path kit 96. For example, a software component provided for configuring the control unit may be programmed to change flow path kit operating parameters from those associated with the first flow path kit to parameters associated with the second flow path kit automatically in accordance with a UID, for example. Such parameters may include timing sequences for operating each of the plurality of valve actuators 102 provided in one or more valve actuation units 100. Such parameters may optionally define process parameters for a specific type of bioprocessing operation (e.g. a liquid chromatography operation) but which are variable between the first and second flow path kits.

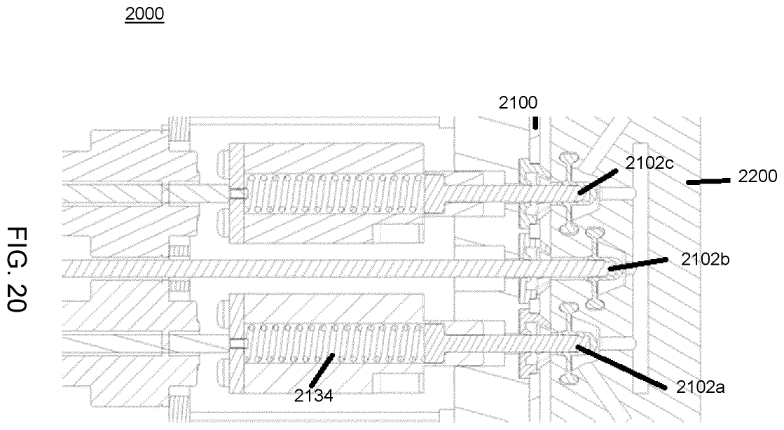

FIG. 20 is a partial cross section of an embodiment of a system 2000 according to the present invention that includes a valve actuation unit 2100 and a membrane valve cassette 2200 coupled thereto.

The system 2000 differs from previous embodiments in that various membranes in the membrane valve cassette 2200 are provided at different depths/operational levels therein. Complex three-dimensional (3D) routing of fluids in the membrane valve cassette 2200 is thus enabled by various embodiments (e.g. by providing fluid routing pathways that are defined through various different depth coplanar levels therein, optionally using various non-angular tubes). Optimal routing of flow paths may thus be provided, e.g. in order to the reduce pressure drop in the membrane valve cassette 2200. Such membrane valve cassettes may also be formed from a plurality of layers, e.g. that are laser welded together.

Additionally, since membranes/valve interfaces may be provided at different depths in the membrane valve cassette 2200, they can effectively be overlapped as viewed in projection from the planar outer surface of the membrane valve cassette 2200. This thus enables a more compact membrane valve cassette 2200 to be provided (e.g. having a smaller overall outer surface area).

In the design shown in FIG. 20, valve actuation unit 2100 is provided with an array of three rods 2102a, 2102b, 2102c. These are driven by respective linear actuators that include respective springs 2134. The two outermost linear actuators that drive rods 2102a and 2102c are provided nearer to the outer mounting surface of the valve actuation unit 2100 than the third linear actuator (not shown). In this way, the centremost rod 2102b passes between the outermost linear actuators 2102a, 2102c, such that a compact design of the valve actuation unit 2100 is also provided. Linear actuators may thus be provided in two or more layers within a valve actuation unit.

The array of rods 2102a, 2102b, 2102c has two thereof (2102a, 2102c) that project from the outer mounting surface of the valve actuation unit 2100 by substantially the same distance. The third rod 2102b projects from the outer mounting surface of the valve actuation unit 2100 by a greater distance.

In various alternative embodiments, however, it is envisaged that an array of rods (e.g. three or more) may be provided that all project from the outer mounting surface of a valve actuation unit by substantially the same distance. In that case, the membrane valve cassette may be adapted to interface to such an arrangement (e.g. by providing extension pins or the like pre-coupled to the membranes or valve interfaces therein). Additionally, or alternatively, one or more intermediate actuator depth adapter(s) (not shown) may be provided for use with (or as part of) the membrane valve cassette. Such an intermediate actuator depth adapter may, for example, be provided between the membrane valve cassette and the outer mounting surface of the valve actuation unit. With such an arrangement a reconfigurable bioprocessing system can be provided with a standardised actuator array, whilst different consumable (e.g. single use) membrane valve cassettes can then be used according to customer need.

Various alternative embodiments may include for the provision of actuators on, for example, two sides of a membrane valve cassette. These may, optionally, include the provision of an L-shaped seat or the like for interfacing to the membrane valve cassette and/or the provision of one or more mobile/repositionable actuator block.

In various embodiments, actuators or parts thereof (e.g. rods) may be provided according to various patterns at the outer mounting surface of the valve actuation unit. Where such patterns include non-rectangular/square array shapes (e.g. quincunx, staggered, hexagonal, triangular, etc.) they can also be made even more compact in design/layout.

Various examples of the present disclosure may be provided in which integration of additional components and/or functionality may be provided (e.g. within a membrane valve cassette). For example, sensing of fluid and/or non-fluid parameters, identification of mounted consumables parts, and/or indicator/display features, etc. may be provided. For example, LEDs, LCDs and/or e-INK displays may be integrated into a membrane valve cassette and can be used to illustrate/display status, information relating to a membrane valve manifold, valve positions, system parameters, fluid flow and/or sensing features, etc. Such additional components and/or functionality may be activated automatically.

Various aspects and embodiments of the present invention thus provide a reconfigurable bioprocessing system that is flexible as well as being easy to use and configure. Such a reconfigurable bioprocessing system may thus reduce the need for skilled/expert/specialised operators as well as being faster and more reliable to use and reconfigure.

Variations or modifications to the systems and methods described herein are set out in the following paragraphs.

In the above examples, specific membrane valve cassettes 200 in the form of configurable flow path cassette 300 and system valve cassette 400 have been described. It will be appreciated that other membrane valve cassettes 200 may also be used with the valve actuation unit 100 described above, to provide a number of different configurable flow paths and bioprocessing operations. The configurable flow path cassette 300 and system valve cassette 400 described above are provided by way of example only.

Other membrane valve cassettes that may be used with the valve actuation unit 100 may include fewer valve interfaces 202. For example, blanking plates may be provided instead of a valve interface. Alternatively, the housing may include voids in which the rods 102 are located when a membrane valve cassette with fewer valve interfaces 202 is fitted to the valve actuation unit 100. Accordingly, in such examples, the number of valve interfaces 202 in a membrane valve cassette may be different to the number of rods 102 in the valve actuation unit 100.

In another example, a smaller sized membrane valve cassette may be used with the valve actuation unit 100. For example, the valve actuation unit 100 may be used with a membrane valve cassette with 6 or 9 valve interfaces incorporated within a housing of shorter length. This may be achieved by fitting the smaller sized membrane valve cassette to four of the protrusions 106 on the valve actuation system 100 (instead of six).

Although the rods 102 and valve interfaces 202 in the above examples are provided in a four by three array (i.e. twelve rods 102 and valve interfaces 202), different numbers and/or configurations of rods and/or valve interfaces may be provided in alternative examples. For example, the rods and/or valve interfaces may be provided in a square array such as a 4×4 array, a 3×3 array or a 2×2 array, or a rectangular array. In a further example, the rods and/or valve interfaces may be provided in an alternative configuration, such as an arrangement of rods and/or valve interfaces comprising a quincunx pattern.

An arrangement comprising a quincunx pattern may be implemented in order to control fluid flow through a manifold provided in a membrane valve cassette. For example, such an arrangement may control fluid flow through a manifold comprising a central conduit connected to two pairs of oppositely opposed laterally extending conduits, where the first pair of oppositely opposed laterally extending conduits is spaced with respect to the second pair of laterally extending conduits along a central axis of the central conduit, and in which the first pair of oppositely opposed laterally extending conduits and the second pair of oppositely opposed laterally extending conduits are substantially parallel with respect to one another. Rods at the periphery of the quincunx pattern may be used to actuate valve interfaces provided in each of the laterally extending conduits, while a rod at the centre of the quincunx pattern may be used to actuate a valve interface provided in the central conduit, thereby allowing fluid flow through the manifold to be controlled. Valve actuators implemented in a quincunx pattern may also be used to control fluid flow through alternative manifolds, such as T-sections.

In various embodiments, an actuator layout pattern and/or one or more manifolds/conduits/channels with a membrane valve cassette may be provided that allow for various fluid conduit orientations to be provided therein. Optionally, membrane valve cassettes may include fewer membrane valves/valve interfaces therein than actuators that are provided on a corresponding actuator plate to which the membrane valve cassette(s) are to be connected in use. This allows even more re-configurability options to be enabled. For example, various embodiments might be provided having at least one manifold/conduit/channel orientated at various angles (e.g. >10, >20, >45, 90, from about 5 to about 45 or 40, from about 10 to 45 or 40, from about 20 to 45 or 40, from about 30 to 45 or 40, from about 5 to about 30, from about 10 to 30, from about 20 to 30, etc. degrees) with respect to the horizontal. Various embodiments may include a plurality of such manifolds/conduits/channels connected together via membrane valves.

To encourage air to be released from the cavity 238 when a corresponding rod 102 is inserted into the cavity 238, a groove may be provided in a side wall of the cavity 238. For example, a groove may be provided in the resiliently deformable collar 244 through which the head portion 124 of the rod 102 is inserted. As another example, the rod 102 itself may additionally or alternatively include a groove to allow for the passage of air. For example, the groove may be provided in the head portion 124 of the rod 102.

In the above examples, the configurable flow path cassette 300 and the system valve cassette 400 have been described as having distinct functionality. In alternative implementations, a common membrane valve cassette may be implemented, having the functionality of both the configurable flow path cassette 300 and the system valve cassette 400. This may be achieved by implementing two or more layers of valve interfaces 202. For example, the functionality of the configurable flow path cassette 300 may be provided in a first layer of a common membrane valve cassette, and the functionality of the system valve cassette 400 may be provided in a second layer of the common membrane valve cassette. Where functionality is provided in multiple layers, the rods may be actuatable to third positions where each rod protrudes further from the surface 104 than in the second position. Alternatively, the rods may be mounted on screw mechanisms that allow the lengths of the rod in the first and second positions to be adjusted.

The rods 102 may also be controllable to provide fluid at increased pressure through one or more conduits by restricting the flow of fluid through the first and second ports 222, 224 of the valve interface 202. This may be achieved by driving the rods 102 to positions that are in between the first and second positions, in order to provide a restriction at one of the ports 222, 224.

Although the flow path kit 96 is described above as including specific flow path components, a generic flow path kit may include one or more of the membrane valve cassettes described above, optionally including any of the additional flow path components of the flow path kit 96 (i.e. pumps, air filter, pH/UV sensor device, and/or tubing to connect the components).

The singular terms “a” and “an” should not be taken to mean “one and only one”. Rather, they should be taken to mean “at least one” or “one or more” unless stated otherwise. The word “comprising” and its derivatives including “comprises” and “comprise” include each of the stated features, but does not exclude the inclusion of one or more further features.

The above implementations have been described by way of example only, and the described implementations are to be considered in all respects only as illustrative and not restrictive. It will be appreciated that variations of the described implementations may be made without departing from the scope of the invention. It will also be apparent that there are many variations that have not been described, but that fall within the scope of the appended claims.

1. A reconfigurable bioprocessing system operable to perform a predetermined set of bioprocessing operations within the bioprocessing system, the bioprocessing system comprising:
a base unit comprising a plurality of valve actuators, wherein at least one of the plurality of valve actuators is configured to releasably engage with a membrane valve cassette that is removably attachable to the base unit, and wherein the at least one of the plurality of valve actuators is actuatable to regulate fluid flow through the membrane valve cassette; and
a control system operable to selectively actuate respective ones of the plurality of valve actuators to provide a valve configuration within the membrane valve cassette to enable the base unit and the membrane valve cassette together to perform one of the predetermined set of bioprocessing operations.

2. The bioprocessing system of claim 1, wherein the predetermined set of bioprocessing operations comprise one or more of: a chromatography operation, a mixing operation and/or a filtration operation.

3. The bioprocessing system of claim 1, wherein the base unit further comprises an attachment mechanism configured to removably attach the membrane valve cassette to the base unit.

4. The bioprocessing system of claim 1, wherein the base unit further comprises a plurality of linear actuators, wherein each of the plurality of valve actuators is configured to be independently actuated by a respective one of the plurality of linear actuators.

5. The bioprocessing system of claim 1, further comprising a casing that houses the base unit, wherein the casing comprises an elongate recess configured to accommodate a chromatography column.

6. A membrane valve cassette for a reconfigurable bioprocessing system according to claim 1, wherein the membrane valve cassette is configured to removably attach to a base unit of the reconfigurable bioprocessing system.

7. The membrane valve cassette of claim 6, further comprising a plurality of conduits and a plurality of valve interfaces, each of the plurality of valve interfaces configured to be actuated by a respective one of the plurality of valve actuators, each valve interface being actuatable between a first configuration in which fluid flow through one of the plurality of conduits is prevented and a second configuration in which fluid flow through the one or the plurality of conduits is enabled.

8. The membrane valve cassette of claim 7, wherein each of the plurality of valve interfaces is movable in a first direction by exertion of a push force on the valve interface by a respective one of the plurality of valve actuators and is movable in a second direction opposite to the first direction by exertion of a pull force on the valve interface by the respective one of the plurality of valve actuators.

9. The membrane valve cassette of claim 7, wherein at least two of the valve interfaces and/or two of the membranes are operable at different depths or coplanar levels therein.

10. The membrane valve cassette of claim 9, further comprising at least one conduit therein that provides for fluid flow in a direction out of a plane substantially parallel to a surface of the membrane valve cassette that interfaces with an outer mounting surface of a valve actuation unit.

11. The membrane valve cassette of claim 7, wherein each of the plurality of valve interfaces comprises a respective resiliently deformable membrane comprising a receiving mechanism configured to releasably engage the respective one of the plurality of valve actuators.

12. The membrane valve cassette of claim 11, wherein the receiving mechanism comprises a cavity comprising an internal collar configured to releasably engage a corresponding portion of the respective one of the plurality of valve actuators to enable the push and pull forces to be exerted on the resiliently deformable membrane.

13. A flow path pressure monitoring cassette for a bioprocessing system, wherein the flow path pressure monitoring cassette is removably attachable to the bioprocessing system, the flow path pressure monitoring cassette comprising:
one or more conduits; and
an attachment mechanism configured to engage a pressure sensing device for sensing a pressure of fluid flowing through at least one of the one or more conduits, wherein the attachment mechanism is configured to seal around a sensing area of the pressure sensing device when the pressure sensing device is engaged with the attachment mechanism.

14. The flow path pressure monitoring cassette of claim 13, wherein the attachment mechanism is further configured to stabilise the engagement of the sensing area of the pressure sensing device with the at least one of the one or more conduits when the pressure sensing device is engaged with the attachment mechanism.

15. A flow path air trap cassette for a bioprocessing system, wherein the flow path air trap cassette is removably attachable to the bioprocessing system, the flow path air trap cassette comprising:
one or more conduits;
an air trap in fluidic communication with at least one of the one or more conduits, wherein the air trap is configured to capture air present in fluid flowing through the at least one of the one or more conduits; and
a sensor configured to detect a level of fluid within the air trap.

16. The flow path air trap cassette of claim 15, wherein the sensor is a first sensor, the flow path air trap cassette further comprising a second sensor, the second sensor configured to detect a second level of fluid within the air trap, wherein the second level of fluid is greater than the first level of fluid.

17. A configurable flow path cassette for a bioprocessing system, wherein the configurable flow path cassette is removably attachable to the bioprocessing system, the configurable flow path cassette comprising:
a plurality of ports comprising a first port, a first group of ports, and a second group of ports, wherein the first group of ports is in fluidic communication with the first port; and
a plurality of valve interfaces that are actuatable to provide a first configuration in which the second group of ports is in fluidic communication with the first port and a second configuration in which the second group of ports is in fluidic communication with the second port.

18. The configurable flow path cassette of claim 17, wherein the plurality of valve interfaces are actuatable to regulate the flow of fluid through each of the first group of ports and/or each of the second group of ports.

19. A conductivity sensing device for a bioprocessing system, the conductivity sensing device comprising:
a conductivity sensor;
a sealing portion having an opening configured to receive the conductivity sensor; and
a casing comprising an attachment mechanism configured to attach the conductivity sensing device to the bioprocessing system;
wherein the sealing portion is configured to form a seal around the conductivity sensor when the conductivity sensing device is attached to the bioprocessing system.

20. The conductivity sensing device of claim 19, wherein the sealing portion is configured to translate a force applied to the sealing portion to the conductivity sensor to form the seal around the conductivity sensor.

21. The conductivity sensing device of claim 20, wherein the attachment mechanism comprises a snap-fit attachment mechanism, and wherein the force is applied to the sealing portion by the snap-fit attachment of the conductivity sensor to the bioprocessing system.

22. A flow path kit for a bioprocessing system, the flow path kit comprising at least one of:
the membrane valve cassette;
the flow path pressure monitoring cassette;
the flow path air trap cassette; and
the configurable flow path cassette;
wherein the flow path kit optionally further comprises the conductivity sensing device of claim 19.
